(19) United States
(12) Patent Application Publication
(10) Pub. No.: US 2025/0249245 A1
(43) Pub. Date: Aug. 7, 2025

(54) HIDDEN COCHLEAR IMPLANT SYSTEM WITH AN IN-CANAL WIRELESS TRANSMISSION

(71) Applicant: SMART SOUND LTD., NIRIT (IL)

(72) Inventor: HAREL ZILBERSHLAG, GIVAT SHMUEL (IL)

(21) Appl. No.: 19/191,047

(22) Filed: Apr. 28, 2025

Related U.S. Application Data
(63) Continuation-in-part of application No. 17/339,380, filed on Jun. 4, 2021, now Pat. No. 12,296,168.
Application No. 17/339,380 is a continuation-in-part of application No. PCT/IB2019/060541, filed on Dec. 8, 2019.
(60) Provisional application No. 62/777,138, filed on Dec. 8, 2018.
(60) Provisional application No. 62/809,663, filed on Feb. 24, 2019.
(60) Provisional application No. 62/859,481, filed on Jun. 10, 2019.

(51) Int. Cl. A61N 1/36; A61N 1/05; A61N 1/372; A61N 1/378

(52) U.S. Cl. CPC A61N 1/36038; A61N 1/0541; A61N 1/37229; A61N 1/3787

(57) ABSTRACT

An in-canal wireless transmitting system (ICWTS) is provided that is configured to be placed within an ear canal of a user and the ear. The ICWTS is associated with an implanted unit that is configured to stimulate an organ in the head of a user, such as an auditory nerve, vestibular system, brain, or a combination thereof. The ICWTS comprises a transmitting antenna configured to transmit electrical energy and receiver antenna configured to receive the electrical energy, wherein the electrical energy comprises data in the form of electrical signals as well as power, that function as power source configured to power the implanted unit through the transmitting antenna.

### CROSS-REFERENCE TO RELATED PATENT APPLICATIONS

This application is a Continuation in Part of pending U.S. patent application Ser. No. 17/339,380, filed Jun. 4, 2021, which is a Continuation in Part of the U.S. National Phase entry of PCT Patent Application No. PCT/IB 2019/060541, filed Dec. 8, 2019, which claims priority to U.S. Provisional Patent Application Ser. No. 62/777,138, filed Dec. 8, 2018, U.S. Provisional Patent Application Ser. No. 62/809,663, filed Feb. 24, 2019, and U.S. Provisional Patent Application Ser. No. 62/859,481, filed Jun. 10, 2019, the entire contents of each of which is incorporated herein by reference in its entirety.

### FIELD OF THE INVENTION

The present subject matter relates to hearing aids. M ore particularly, the present subject matter relates to hearing aids of the cochlear implants.

### BACKGROUND OF THE INVENTION

A hearing aid is a device designed to improve hearing by making sound audible to a person with hearing loss. Among various types of hearing aids currently available, cochlear implants are of interest to the present subject matter.

A cochlear implant is a surgically implanted neuroprosthetic device configured to provide a person with moderate to profound sensorineural hearing loss a modified sense of sound. Cochlear implants bypass the normal acoustic hearing process to replace it with electric signals that directly stimulate the auditory nerve. A user of a cochlear implant can, after intensive auditory training, learn to interpret the signals stimulating the auditory nerve as sound and speech.

FIG. 1 schematically illustrates, according to an exemplary embodiment, a perspective view of internal and external components of a human ear and a prior art cochlear implant implanted in the human ear.

The components of the human ear 9 that are relevant to the present subject matter and prior art are the pinna 910, the cochlea 920, the auditory nerve 930, the ear canal 940, and the ear drum 950.

The prior art cochlear implant 3 comprises an external microphone 310 configured to receive sound signals, convert the sound signals to electrical sound signals, and transmit electrical energy and data, for example the aforementioned electrical sound signals. The external microphone 310 can be attached to the pinna 910, as shown in FIG. 1. However, it is also possible to attach the external microphone 310 to any item convenient to a user of the prior art cochlear implant 3. The external microphone 310 is electrically connected to an external modulator 320, configured to receive electrical sound signals from the external microphone 310, modulate a carrier signal with the electrical sound signals to produce a modulated electrical carrier signal, and transmit the modulated electrical carrier signal. A common carrier signal that is used in prior art cochlear implants 3 is a radio frequency signal, known as RF signal. The external modulator 320 is electrically connected to an external transmitting antenna 330. The external transmitting antenna 330 is configured to receive the modulated electrical carrier signal from the external modulator 320, convert the modulated electrical carrier signal to a modulated wireless carrier signal, and wirelessly transmit electrical energy and data, for example the aforementioned modulated wireless carrier signal. A common wireless carrier signal that is used in prior art cochlear implants is an electromagnetic RF signal. The external transmitting antenna 330 normally has a coil-like shape. All, the external microphone 310, the external modulator 320, and the external transmitting antenna 330 are external components of the prior art cochlear implant 3. In other words, the external microphone 310, the external modulator 320, and the external transmitting antenna 330 are attached to the skin of the user. However, alternatively, the external transmitting antenna 330 can still be attached to the skin, while the external microphone 310, and alternatively the external modulator 320, can be attached to a clothing of the user. Additional components can be connected to the external components of the prior art cochlear implant 3, in order to facilitate their function. These components include electronics, for example digital signal processor (DSP) chips the selectively filter the sound signals received by the external microphone 310 to prioritize audible speech; and a battery.

The prior art cochlear implant 3 further comprises implanted components, that is components that are implanted under a skin, or in the internal parts of the ear 9, of the user. Thus, the prior art cochlear implant 3 further comprises an internal receiving antenna 340 configured to receive electrical energy and data, for example the aforementioned modulated wireless carrier signal from the external transmitting antenna 330, convert the modulated wireless carrier signal to a modulated electrical carrier signal and transmit electrical energy and data, for example the aforementioned modulated electrical carrier signal. The internal receiving antenna 340 can also have a coil-like structure, and it is implanted under the skin, normally under just above the pinna 910, on the mastoid bone. The prior art cochlear implant 3 further comprises an external magnet 350 placed adjacent to the external transmitting antenna 330 and an internal magnet 360 placed adjacent to the internal receiving antenna 340. The purpose of the external magnet 350 and the internal magnet 360 is to facilitate placement of the external transmitting antenna 330 on the skin in vicinity of the internal receiving antenna 340 that is implanted under the skin, on the mastoid bone. The internal receiving antenna 340 is electrically connected to an internal processor 370, configured to receive the modulated electrical carrier signal from the internal receiving antenna 340, demodulate the modulated electrical carrier signal to produce electrical sound signals, and transmit the electrical sound signals to an electrode array 380. According to one embodiment, the internal processor 370 is in a form of a chip, for example an ASIC chip. Thus, the internal processor 370 is further configured to perform additional tasks relating to controlling the function of the prior art cochlear implant 3, and ensuring proper function of the prior art cochlear implant 3. Some exemplary functions of the internal processor 370 include error check of decoded electrical sound signals to ensure proper decoding, controlling the timing and direction of transmission of the decoded electrical sound signals, and the like. Another implanted component of the prior art cochlear implant 3 is an electrode array 380 that is implanted in the cochlea 920 of the ear 9. The internal processor 370 is electrically connected to the electrode array 380. The electrode array 380 is configured to receive electrical sound signals from the internal processor 370, and stimulate with these electrical sound signals the auditory nerve 930, that is adjacent to the cochlea 920. Then, the auditory nerve 930 transmits the signals that it receives from the electrode array 380 to the brain, and the brain translates these signals to a sense of sound and speech.

One drawback of the prior art cochlear implant 3 is that it includes external parts, as detailed above. There are users that fill annoyed from carrying such external components, whether on their ear's pinna 910, or on a clothing, and on the skin in the vicinity of the pinna 910. Other users can prefer hiding their hearing impairment, and therefore even prefer not to use the prior art cochlear implant 3, and leave their hearing impairment without treatment.

### SUMMARY OF THE INVENTION

According to an embodiment of the present subject matter, it is provided an in-canal wireless transmitting system (ICWTS) associated with an implant that is configured to stimulate an organ in a head of a user having an ear and an ear canal, the ICWTS comprising: a canal transmitting antenna to be positioned within the ear canal, wherein the canal transmitting antenna is configured to transmit electrical energy; and an implanted receiving antenna to be implanted in the ear, wherein the implanted receiving antenna is connected to the implant and receives the electrical energy that functions as a power source.

In accordance with another embodiment of the present subject matter, the ICWTS is further comprising a microphone and wherein the electrical energy further comprises sound signals received by the implanted receiving antenna and the organ is an auditory nerve.

In accordance with another embodiment of the present subject matter, the implanted receiving antenna is positioned in a mastoid cavity in the ear, in a close proximity to an inner wall of the ear canal that is facing a direction of the ear canal.

In accordance with another embodiment of the present subject matter, the canal transmitting antenna is directed to the implanted receiving antenna in the mastoid cavity.

In accordance with another embodiment of the present subject matter, the canal transmitting antenna is transmitting unidirectional wireless transmission of electrical energy and bidirectional wireless transmission of data communication.

In accordance with another embodiment of the present subject matter, the data communication is modulated on a power carrier transmission that transmits power.

In accordance with yet another embodiment of the present subject matter, a hidden transmission system that comprises the ICWTS as described and an external transmission system comprising an additional receiving antenna provided to the implant and located in a mastoid cavity facing an outer side of the head and an external transmitting antenna located on the outer side, wherein the additional receiving antenna receives power and data from the external transmission antenna.

In accordance with another embodiment of the present subject matter, the hidden transmission system further comprises a bone anchored hearing aid.

In accordance with yet another embodiment of the present subject matter, a fully implantable cochlear implant configured to be combined with the ICWTS so as to form a combined hidden transmission system, wherein the combined hidden transmission system comprises:
a processor;
a microphone functions as a main data source and collects sound;
a power source;
an electrode array;
   wherein the electrical energy transmitted from the canal transmitting antenna functions as alternative power supply or as a charger to the power source and the data is transmitted from the canal transmitting antenna to the processor through the receiving antenna.

In accordance with another embodiment of the present subject matter, the system further comprises an external transmission system comprising an additional receiving antenna provided to the fully implantable cochlear implant and located in a mastoid cavity facing an outer side of the head and an external transmitting antenna located on the outer side of the head, wherein the additional receiving antenna receives power and data from the external transmission antenna.

A method of positioning the ICWTS is also described wherein the method comprises implanting the receiving antenna within a mastoid cavity in close proximity to an inner wall of the ear canal facing the ear canal, and placing the transmitting antenna in the ear canal and directing the transmitting antenna towards the receiving antenna.

In accordance with another embodiment of the present subject matter, the receiving antenna is configured to be implanted within a tragus of the ear and the transmitting antenna is configured to be placed within the ear canal directed towards the receiving antenna.

In accordance with another embodiment of the present subject matter, the transmitting antenna is designed as a ring and is pierced so as to interconnect with the receiving antenna.

In accordance with another embodiment of the present subject matter, the canal transmitting antenna and the receiving antenna are coils that function as inductive coils system transferring electrical energy and data using an electromagnetic transmission.

In accordance with another embodiment of the present subject matter, the canal transmitting antenna is housed within a canal unit that is provided with an adjustment mechanism by which the position of the transmission antenna can be adjusted manually.

In accordance with another embodiment of the present subject matter, an external unit (BTE) comprises a plurality of socket connections configured to receive at least two transmission coils while the BTE can be switched from sending signals of one of the at least two transmission coils at a time.

In accordance with another embodiment of the present subject matter, the one of the transmitting coils is a canal transmitting coil that is configured to be plugged in one of the plurality of sockets.

Unless otherwise defined, all technical and scientific terms used herein have the same meaning as commonly understood by one of ordinary skill in the art to which this subject matter belongs. Although methods and materials similar or equivalent to those described herein can be used in the practice or testing of the present subject matter, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Embodiments are herein described, by way of example only, with reference to the accompanying drawings. With specific reference now to the drawings in detail, it is stressed that the particulars shown are by way of example and for purposes of illustrative discussion of the preferred embodiments, and are presented in the cause of providing what is believed to be the most useful and readily understood description of the principles and conceptual aspects of the embodiments. In this regard, no attempt is made to show structural details in more detail than is necessary for a fundamental understanding, the description taken with the drawings making apparent to those skilled in the art how several forms may be embodied in practice.

In the drawings:

FIG. 1 schematically illustrates, according to an exemplary embodiment, a perspective view of internal and external components of a human ear and a prior art cochlear implant implanted in the human ear.

FIG. 2 schematically illustrates, according to an exemplary embodiment of the present subject matter, a perspective see-through view of a canal unit of a hidden cochlear implant system.

FIG. 3 schematically illustrates, according to an exemplary embodiment of the present subject matter, a perspective see-through view of a canal unit of a hidden cochlear implant system, further comprising a handle.

FIG. 4 schematically illustrates, according to an exemplary embodiment of the present subject matter, a perspective see-through view of a canal unit of a hidden cochlear implant system, further comprising an attractable element.

FIG. 5 schematically illustrates, according to an exemplary embodiment of the present subject matter, a perspective see-through view of a canal unit of a hidden cochlear implant system, further comprising a handle and an attractable element.

FIG. 6 schematically illustrates, according to an exemplary embodiment of the present subject matter, a perspective see-through view of a canal unit of a hidden cochlear implant system, further comprising a canal optical transmitter instead of the canal transmitting antenna.

FIG. 7A schematically illustrates, according to an exemplary embodiment of the present subject matter, a perspective see-through view of a canal unit of a hidden cochlear implant system divided to two parts.

FIG. 7B schematically illustrates, according to an exemplary embodiment of the present subject matter, a perspective see-through view of a canal unit of a hidden cochlear implant system divided to two parts that are partially reassembled.

FIG. 8 schematically illustrates, according to an exemplary embodiment of the present subject matter, a perspective view of internal and external components of a human ear and a canal unit inserted in the ear canal.

FIG. 9 schematically illustrates, according to an exemplary embodiment of the present subject matter, a side view of an implanted unit of a hidden cochlear implant system, the implanted unit comprising a processor that is configured to be implanted in the vicinity of the cochlea.

FIG. 10 schematically illustrates, according to an exemplary embodiment of the present subject matter, a perspective view of internal components of a human ear and an implanted unit implanted in the vicinity of the cochlea, the implanted unit comprising a processor that is configured to be implanted in the vicinity of the cochlea.

FIG. 11 schematically illustrates, according to an exemplary embodiment of the present subject matter, a perspective view of an implanted unit comprising a processor that is configured to be implanted under the skin, in the vicinity of the pinna, on the mastoid bone.

FIG. 12 schematically illustrates, according to an exemplary embodiment of the present subject matter, a perspective view of internal components of a human ear and an implanted unit implanted in the vicinity of the cochlea, the implanted unit comprising a processor that is configured to be implanted under the skin, in the vicinity of the pinna, on the mastoid bone.

FIG. 13 schematically illustrates, according to an exemplary embodiment of the present subject matter, a side view of another embodiment of an implanted unit of a hidden cochlear implant system.

FIG. 14 schematically illustrates, according to an exemplary embodiment of the present subject matter, a perspective view of internal components of a human ear and another embodiment of an implanted unit that is implanted partially in the vicinity of the cochlea, and partially implanted under the skin, in the vicinity of the pinna, on the mastoid bone.

FIG. 15 schematically illustrates, according to an exemplary embodiment of the present subject matter, a perspective see-through view of a side view of an implanted unit of a hidden cochlear implant system, further comprising a cochlear optical receiver instead of the cochlear receiving antenna.

FIG. 16 schematically illustrates, according to an exemplary embodiment of the present subject matter, a perspective view of internal components of a human ear and an implanted unit implanted partially in the vicinity of the cochlea, and partially implanted under the skin, in the vicinity of the pinna, on the mastoid bone, as well as a canal unit inserted in the ear canal.

FIG. 17 schematically illustrates, according to an exemplary embodiment of the present subject matter, a side view of an internal receiving antenna adaptor electrically connected to a cochlear receiving antenna.

FIGS. 18-20 schematically illustrate exemplary embodiments, in accordance with the present subject matter, of a storing member.

FIG. 21 schematically illustrates, according to an exemplary of the present subject matter, a side view of another embodiment of an implanted unit of a hidden cochlear implant system further comprising a ground.

FIG. 22 schematically illustrates, according to an exemplary of the present subject matter, a side view of another embodiment of an implanted unit of a hidden cochlear implant system further comprising an implant electrical power source.

FIG. 23A schematically illustrates, according to an embodiment of the present subject matter, a view of a canal unit of the hidden cochlear implant system.

FIG. 23B schematically illustrates, according to another embodiment of the present subject matter, a partial view of a canal unit of the hidden cochlear implant system.

FIG. 24 schematically illustrates, according to another embodiment of the present subject matter, an implant system that some of the implant system components are configured to reside in an external unit.

FIG. 25 schematically illustrates, according to an embodiment of the present subject matter, a view of a receiving antenna.

FIG. 26 schematically illustrates, according to an exemplary embodiment of the present subject matter, a perspective view of an implanted unit comprising a processor that is configured to be implanted under the skin, in the vicinity of the pinna, on the mastoid bone.

FIG. 27 schematically illustrates, according to an embodiment of the present subject matter, a view of an exemplary conduit.

FIG. 28 schematically illustrates, according to an exemplary embodiment of the present subject matter, a perspective view of internal components of a human ear and an implanted unit when the electrode array is implanted in the cochlea, and the coils and processor are implanted in the ear, in the mastoid cavity, as well as a canal unit inserted in the ear canal.

FIG. 29 schematically illustrates, according to an exemplary embodiment of the present subject matter, a perspective see-through view of a canal unit of a hidden cochlear implant system.

FIG. 30 schematically illustrates, according to an exemplary embodiment of the present subject matter, a perspective view of an implanted unit to be partially implanted within the mastoid cavity.

FIG. 31A schematically illustrates, according to an exemplary embodiment of the present subject matter, a perspective view of an outer ear with an implanted unit and a receiving coil positioned in the vicinity of the tragus.

FIG. 31B schematically illustrates the perspective view shown in FIG. 31A, with a pierced transmitting coil.

FIG. 31C schematically illustrates, according to another exemplary embodiment of the present subject matter, a perspective view of an outer ear with an implanted unit and a receiving coil positioned in the helix with a pierced transmitting coil.

FIG. 32A schematically illustrates, according to an exemplary embodiment of the present subject matter, a perspective view of an ICWTS incorporated within a BAHA partially implanted within an ear.

FIG. 32B schematically illustrates, according to an exemplary embodiment of the present subject matter, an ICWTS implanted unit incorporated within a BAHA Implant.

FIG. 33 schematically illustrates a fully implantable unit, according to an exemplary embodiment of the present subject matter.

FIG. 34A schematically illustrates a BTE device configured to be connected to an ICWTS and an external transmission system, according to an exemplary embodiment of the present subject matter.

FIG. 34B schematically illustrates a BTE device connected to an ICWTS and an external transmission system, according to an exemplary embodiment of the present subject matter.

### DESCRIPTION OF THE PREFERRED EMBODIMENTS

Before explaining at least one embodiment in detail, it is to be understood that the subject matter is not limited in its application to the details of construction and the arrangement of the components set forth in the following description or illustrated in the drawings. The subject matter is capable of other embodiments or of being practiced or carried out in various ways. Also, it is to be understood that the phraseology and terminology employed herein is for the purpose of description and should not be regarded as limiting. In discussion of the various figures described herein below, like numbers refer to like parts. The drawings are generally not to scale.

For clarity, non-essential elements were omitted from some of the drawings.

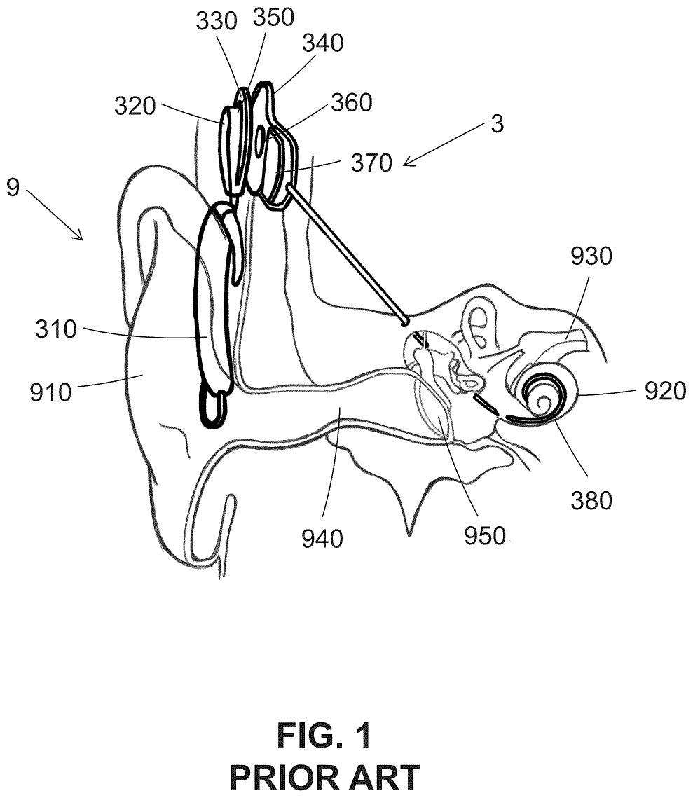

Referring now to FIG. 1 schematically illustrating, according to an exemplary embodiment, a perspective view of internal and external components of a human ear and a prior art cochlear implant implanted in the human ear. The present subject matter provides a cochlear implant that does not comprise external components attached to external parts of a user, for example, the user's pinna 910, a clothing of the user, and on the skin of the user in the vicinity of the pinna 910.

The present subject matter further provides, according to some embodiments, a cochlear implant that does not comprise components that are implanted under the skin, for example in the vicinity of the pinna 910, and from the area of the pinna 910 towards the cochlea 920.

The present subject matter provides in addition, a cochlear implant that can still be used by users that already have implanted parts of a prior art cochlear implant 3, for example, users that have an internal receiving antenna 340, an internal processor 370, and an electrode array 380, implanted under the skin in the area of the pinna 910, on the mastoid bone, in the cochlea 920, and in-between.

In order to distinguish between the cochlear implant of the present subject matter and the prior art cochlear implant 3, the cochlear implant of the present subject matter is designated hereinafter “hidden cochlear implant system”.

According to one embodiment of the present subject matter, components of the hidden cochlear implant system 1 are made of at least one biocompatible material. According to another embodiment, at least components or parts of the hidden cochlear implant system 1 that are exposed to a biological tissue, for example a biological tissue of an ear, are biocompatible. The importance of these embodiments is that since components of the hidden cochlear implant system 1 are configured to be implanted in internal parts of the ear 9, like the cochlea 920, or inserted into the ear canal 940, at least the components or parts of the hidden cochlear implant system 1 that are exposed to a biological tissue have to be made of at least one biocompatible material in order to avoid rejection response to the hidden cochlear implant system 1, inflammation, and the like. Some exemplary biocompatible material of which components or parts of the hidden cochlear implant system 1 can be made, include, but not limited to, biocompatible metals such as stainless steel, cobalt alloys, titanium alloys, and the like; biocompatible ceramics such as aluminum oxide, zirconia, calcium phosphates, and the like; biocompatible polymers such as silicones, poly ethylene, poly vinyl chloride, polyurethanes, polylactides and the like; and biocompatible natural polymers such as collagen, gelatin, elastin, silk, polysaccharides, and the like. It should be noted that this list of biocompatible materials of which components or parts of the hidden cochlear implant system 1 can be made should not be considered a limiting the scope of the present subject matter, but rather to serve only as an exemplary list of biocompatible materials.

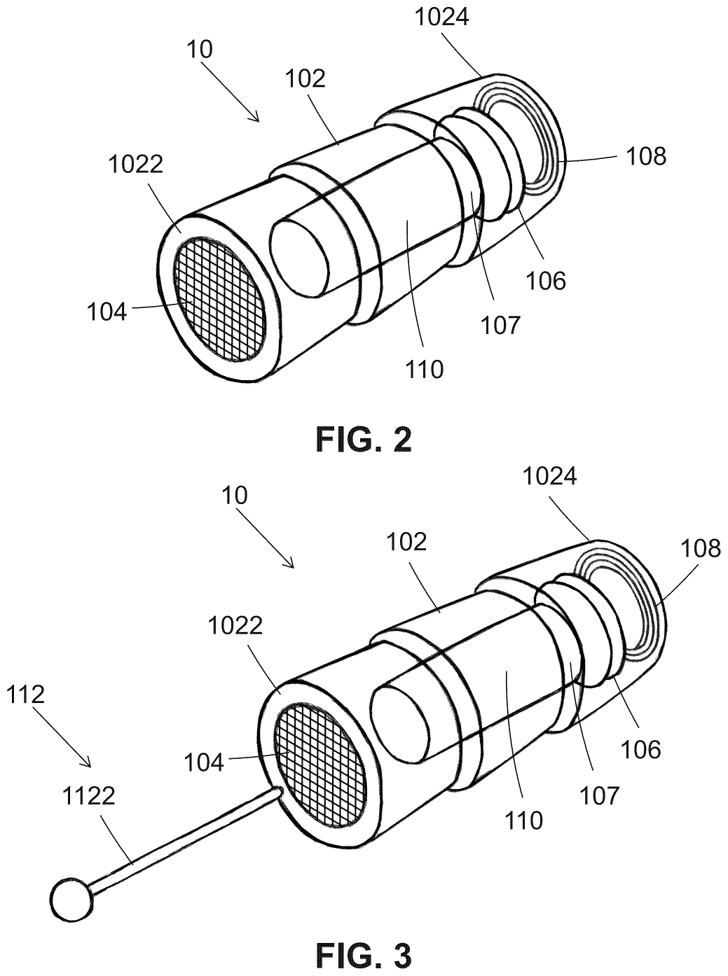

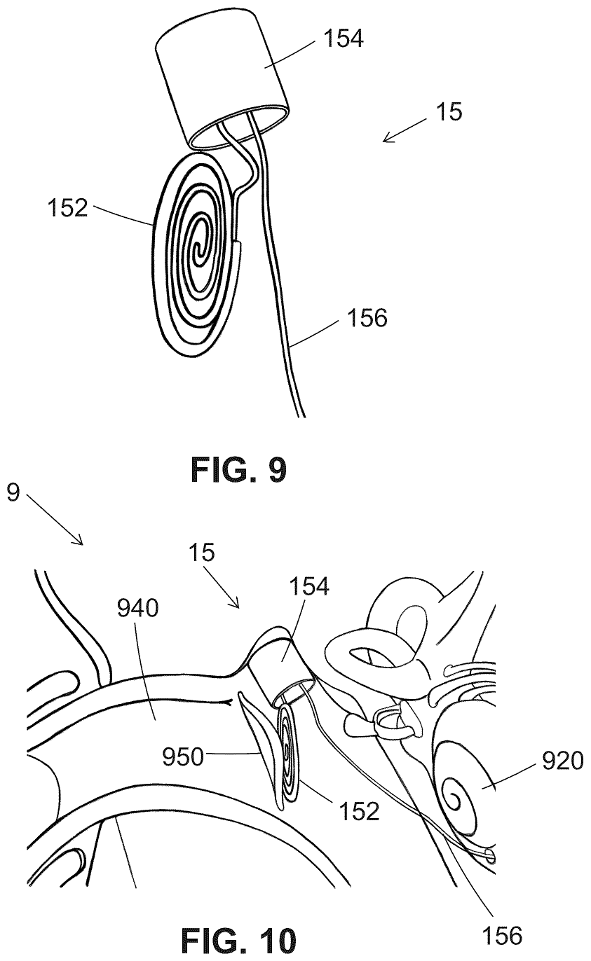

According to one embodiment, the hidden cochlear implant system 1, shown as a whole in FIG. 16, comprises a canal unit 10, shown in FIGS. 2-8, and an implanted unit 15, shown in FIGS. 9-15.

Referring now to FIG. 2 schematically illustrating, according to an exemplary embodiment of the present subject matter, a perspective see-through view of a canal unit of a hidden cochlear implant system.

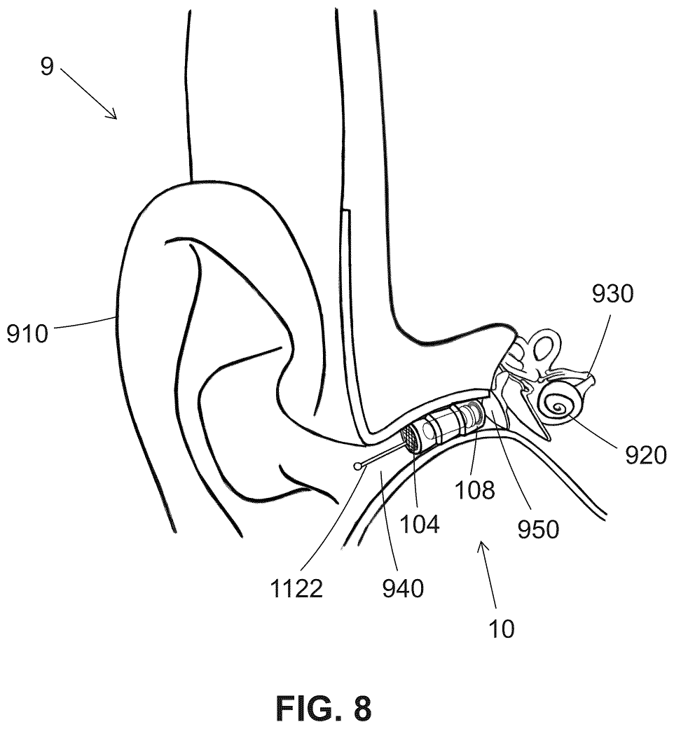

According to one embodiment, the canal unit 10 is configured to be inserted into an ear canal 940 of an ear 9 (as shown in FIG. 8), wherein the canal unit 10 is further configured to receive sound signals that enter into the ear canal 940, convert the sound signals to electrical sound signals, modulate a carrier wave with the electrical sound signals to obtain a modulated carrier wave, and wirelessly transmit electrical energy and data, for example the aforementioned modulated carrier wave. The canal unit 10 is inserted into an ear canal 940 during a surgical procedure.

According to one embodiment, at least part of the implanted unit 15 is configured to be implanted in a vicinity to a cochlear 920 of an ear 9. During such surgical procedure, in order to allow a large space for the implanted unit 15 in the middle ear, the tiny bones called the Malleus, Incus and Stapes can be removed. According to another embodiment, the implanted unit 15 is configured to receive electrical energy and data, for example the aforementioned modulated carrier wave that is transmitted by the canal unit 10, demodulate the modulated carrier wave to obtain electrical sound signals, and stimulate an auditory nerve 930 with the electrical sound signals.

According to one embodiment, the canal unit 10 replaces the external components of the prior art cochlear implant 3 of FIG. 1, which were described above. In other words, usage of the canal unit 10 can negate usage of the external components of the prior art cochlear implant 3. For example, the canal unit 10 replaces the prior art external microphone 310, the prior art external modulator 320, the prior art external transmitting antenna 330, the prior art external magnet 350, the prior art electronics, such as the prior art digital signal processor (DSP) chips, and any other external component that can be part of the prior art cochlear implant 3. A person skilled in the art would appreciate the benefits and advantages of the hidden cochlear implant system 1 of the present subject matter over the prior art cochlear implant 3. Firstly, the hidden cochlear implant system 1 does not comprise external components that are easily visible. Thus, a user can use the hidden cochlear implant system 1 without any one to notice that. This is extremely beneficial and advantageous, especially for people that would not use a prior art cochlear implant 3 because of the annoyance related to carrying external components.

According to one embodiment, the shape and size of the canal unit 10 is configured to adapt to a shape and size of an ear canal 940 of a user. According to another embodiment, the canal unit 10 is elastic so it could adapt its shape and size to the shape and size of the ear canal 940 of the user.

According to one embodiment, the canal unit 10 has a hollow cylindrical shape comprising an outward side 1022 and an inward side 1024. The outward side 1022 of the canal unit 10 is configured to point towards the pinna 910 of the ear 9, namely in an outward direction relative to the ear canal 940, when the canal unit 10 resides in the ear canal 940. The inward side 1024 of the canal unit 10 is configured to point toward an inner part of the ear, more particularly, toward the ear drum 950 and the cochlea 920, when the canal unit 10 resides in the ear canal 940. According to another embodiment, components of the canal unit 10, detailed hereinafter, are configured to be accommodated in an inner space formed by the hollow cylindrical shape of the canal unit 10.

According to one embodiment, the canal unit 10 comprises at least one canal microphone 104, a canal modulator 106, a canal transmitting antenna 108, and a canal electrical power source 110, wherein the at least one canal microphone 104 is electrically connected to the canal modulator 106, the canal modulator 106 is electrically connected to the canal transmitting antenna 108, and the canal electrical power source 110 is electrically connected to any component of the canal unit 10 that requires supply of electrical power, for example the at least one canal microphone 104, the canal modulator 106, and the like.

According to one embodiment, the at least one canal microphone 104 is configured to receive sound signals, convert the sound signals to electrical sound signals, and transmit the electrical sound signals to the canal modulator 106. According to another embodiment, the at least one canal microphone 104 is configured to be placed at the outward side 1022 of the canal unit 10, so the at least one canal microphone 104 would be able to receive sound signals that enter into the ear canal 940.

According to one embodiment, the canal modulator 106 is configured to receive electrical sound signals from the at least one canal microphone 104, modulate an electrical carrier signal with the electrical sound signals to produce a modulated electrical carrier signal, and transmit the modulated electrical carrier signal to the canal transmitting antenna 108.

According to one embodiment, the canal transmitting antenna 108 is configured to receive the modulated electrical carrier signal from the canal modulator 106, convert the modulated electrical carrier signal to a modulated wireless carrier signal, and wirelessly transmit electrical energy and data, for example the aforementioned modulated wireless carrier signal. The modulated wireless carrier signal that is wirelessly transmitted is used to transmits the sound data that is collated by the microphones. The electrical energy that is wirelessly transmitted is used as power to drive the electrical components of the implant, and practically act as a power source for the implanted unit. Preferably, the transmission is an electromagnetic transmission using two coils as will be discussed hereinafter. The data transmission can be modulated on the power carrier transmission. According to another embodiment, the canal transmitting antenna 108 is configured to wirelessly transmit any type of a wireless carrier signal known in the art, for example an electromagnetic RF signal, short-wavelength ultra-high frequency, a technology known as “Bluetooth”, an optical carrier signal, a combination thereof, and the like. The electromagnetic coils transmission can also be referencing as inductive coil system. According to one embodiment, mentioned above, the canal transmitting antenna 108 is configured to transmit electrical energy and data, for example a modulated wireless carrier signal. According to another embodiment, the canal transmitting antenna 108 is configured to transmit electrical energy, for example in a form of a non-modulated wireless carrier signal, namely a carrier signal only. According to one embodiment, the canal transmitting antenna 108 can have any shape known in the art. According to a preferred embodiment, the canal transmitting antenna 108 has a coil-like shape. According to another preferred embodiment, the canal transmitting antenna 108 is configured to be placed at the inward side 1024 of the canal unit 10, so the canal transmitting antenna 108 would be able to transmit electrical energy, for example in a form of a wireless carrier signal, and data, for example a modulated wireless carrier signal, toward the ear drum 950 and the cochlea 920 as will be described hereinafter.

According to one embodiment, the canal transmitting antenna 108 can further comprise a ferromagnetic ferrite in order to improve the efficiency of transmission of the canal transmitting antenna 108.

It is important to notice the use of electromagnetic energy transmission and its function as power source for the implant component instead of placement of a battery in the implanted unit. There are several advantage to this approach such preventing the need of replacement of implanted battery with an additional surgery, preventing the need to remove the battery in MRI testing, and also preventing the need for distant charging of the battery when the battery is wasted.

According to one embodiment, the canal unit 10 can further comprise a canal processor 107, wherein the at least one canal microphone 104 is electrically connected to the canal processor 107, and the canal processor 107 is electrically connected to the canal modulator 106. According to another embodiment, the canal processor 107 is configured to receive electrical sound signals from the at least one canal microphone 104, process the electrical sound signals to produce processed electrical sound signals, and transmit the processed electrical sound signals to the canal modulator 106. An exemplary process of the electrical sound signals that can be performed by the canal processor 107 is selective filtering of electrical sound signals to prioritize electrical sound signals originating from audible speech. Any type of processor that can perform the required processing of electrical sound signals can serve as a canal processor 107. According to a preferred embodiment, the canal processor 107 comprises DSP chips. Another exemplary canal processor 107 is a completely in canal (CIC).

As mentioned above, the canal unit 10 comprises a canal electrical power source 110. Any type of electrical power source known in the art can serve as a canal electrical power source 110. For example, the canal electrical power source 110 can be a battery, a rechargeable battery, and the like. According to a preferred embodiment, the canal electrical power source 110 is a rechargeable battery.

According to one embodiment, the canal unit 10 further comprises a casing 102 configured to accommodate components of the canal unit 10, for example the at least one canal microphone 104, the canal modulator 106, the canal transmitting antenna 108, the canal electrical power source 110, the canal processor 107, and the like. According to another embodiment, the casing 102 is configured to determine the cylindrical shape of the canal unit 10. According to yet another embodiment, the casing 102 is configured to protect the components that are accommodated in the casing 102.

According to one embodiment, the casing 102 has a hollow elongated shape defining a space. According to another embodiment, components of the canal unit 10 are configured to be accommodated in the space of the casing 102. According to yet another embodiment, the case 102 further comprises an outward side 1022 and an inward side 1024. The outward side 1022 of the casing is configured to point towards the pinna 910 of the ear 9, namely in an outward direction relative to the ear canal 940, when the canal unit 10 resides in the ear canal 940. The inward side 1024 of the casing 102 is configured to point toward an inner part of the ear, more particularly, toward the ear drum 950 and the cochlea 920. According to some embodiments, the canal unit 10 is configured to be inserted into the ear canal 940 until the inward side 1024 of the casing 102 is in close vicinity to the ear drum 950, as can be seen for example in FIG. 8 hereinafter.

According to one embodiment, the casing 102, accommodating components of the canal unit 10, is configured to be inserted into the ear canal 940 of a human ear 9. According to yet another embodiment, the shape and size of the casing 102 is configured to adapt to a shape and size of an ear canal 940 of a user. According to still another embodiment, the casing 102 is elastic so it could adapt its shape and size to the shape and size of the ear canal 940 of the user. Thus, the casing 102 is made of any material known in the art that is elastic, for example soft plastic, fabric, silicon and the like, in addition to being made of at least one compatible material, according to some embodiments, as described above. According to a preferred embodiment, the casing 102 is made of silicon. According to a further embodiment, the casing 102 is substantially cylindrical similarly to the substantial cylindrical shape of the ear canal 940.

According to one embodiment, the canal unit 10 can further comprise at least one grasping element 112 configured to facilitate grasping of the canal unit 10, for example during handling of the canal unit 10, insertion of the canal unit 10 into the ear canal 940, or removal of the canal unit 10 from the ear canal 940. Any type of component that is configured to facilitate grasping of the canal unit 10 is under the scope of the present subject matter. Here is a description of some exemplary components that are configured to facilitate grasping of the canal unit 10.

Referring now to FIG. 3 schematically illustrating, according to an exemplary embodiment of the present subject matter, a perspective see-through view of a canal unit of a hidden cochlear implant system, further comprising a handle.

According to one embodiment, the handle 1122 is configured to be grasped, for example during handling of the canal unit 10, insertion of the canal unit 10 into the ear canal 940, or during removal of the canal unit 10 from the ear canal 940. According to another embodiment, the handle 1122 is configured to be grasped by a tool used for handling the canal unit 10, inserting the canal unit 10 into the ear canal 940, or removing the canal unit 10 from the ear canal 940. For example, the handle 1122 is configured to be grasped by tweezers, forceps, fingers of a user, and the like. According to a further embodiment, the handle 1122 is positioned at any place on the canal unit 10 that is suitable for fulfillment of the purpose of using the handle 1122. According to a preferred embodiment, illustrated for example in FIG. 3, the handle 1122 is attached to the outward side 1022 of the canal unit 10. This position of the handle 1122 is preferable because it allows grasping of the canal unit 10 during insertion of the canal unit 10 into the ear canal 940, or during removal of the canal unit 10 from the ear canal 940.

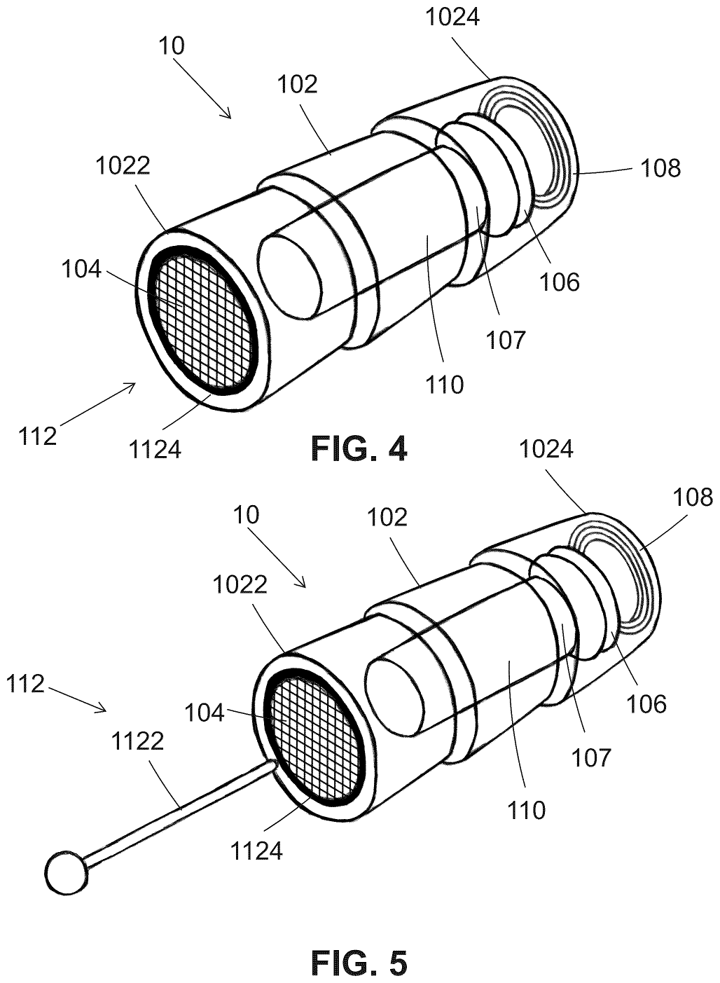

Referring now to FIG. 4 schematically illustrating, according to an exemplary embodiment of the present subject matter, a perspective see-through view of a canal unit of a hidden cochlear implant system, further comprising an attractable element.

According to one embodiment, the attractable element 1124 is configured to be attracted by employing a magnetic force, for example during handling of the canal unit 10, insertion of the canal unit 10 into the ear canal 940, or during removal of the canal unit 10 from the ear canal 940. Thus, according to another embodiment, the attractable element 1124 is made of any material known in the art that is attractable by a magnet, for example iron, nickel, cobalt, gadolinium, dysprosium, and alloys comprising the same. According to yet another embodiment, the attractable element 1124 is magnetic. Thus, the attractable element 1124 is configured to be attracted by any tool known in the art that comprises a magnet, or comprises a material that is attractable by a magnet, and is further suitable for handling the canal unit 10, inserting the canal unit 10 into the ear canal 940, or removing the canal unit 10 from the ear canal 940. According to a further embodiment, the attractable element 1124 can be a piece of a material that is attractable by a magnet, or a magnetic material. According to yet a further embodiment, the attractable element 1124 is positioned at any place on the canal unit 10 that is suitable for fulfillment of the purpose of using the attractable element 1124. According to a preferred embodiment, illustrated for example in FIG. 4, the attractable element 1124 is positioned at the outward side 1022 of the canal unit 10. This position of the attractable element 1124 is preferable because it allows grasping of the canal unit 10 during insertion of the canal unit 10 into the ear canal 940, or during removal of the canal unit 10 from the ear canal 940. According to another preferred embodiment, the attractable element 1124 can have a ring-like shape, as can be seen, for example, in FIG. 4.

Referring now to FIG. 5 schematically illustrating, according to an exemplary embodiment of the present subject matter, a perspective see-through view of a canal unit of a hidden cochlear implant system, further comprising a handle and an attractable element.

According to one embodiment, the canal unit 10 comprises multiple components configured to facilitate grasping of the canal unit 10, for example during handling of the canal unit 10, insertion of the canal unit 10 into the ear canal 940, or removal of the canal unit 10 from the ear canal 940. Thus, as can be seen in FIG. 5, the canal unit 10 can further comprise the handle 1122 as described above and shown in FIG. 3, and the attractable element 1124 as described above, and shown in FIG. 4.

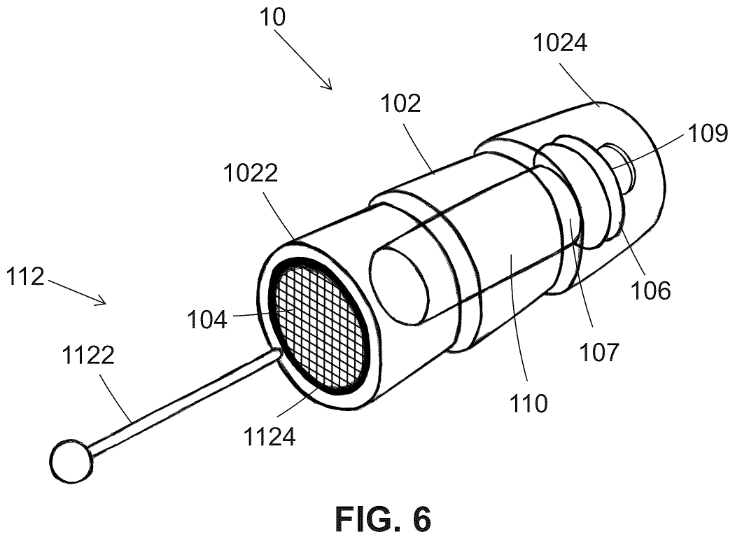

Referring now to FIG. 6 schematically illustrating, according to an exemplary embodiment of the present subject matter, a perspective see-through view of a canal unit of a hidden cochlear implant system, further comprising a canal optical transmitter instead of the canal transmitting antenna. It should be noted that the drum 950 is transparent and allows passage of light therethrough.

As described above, the canal transmitting antenna 108 is configured to transmit electrical energy, for example in a form of wireless carrier signal, and data, for example in a form of a modulated wireless carrier signal. Any type of wireless transmission of electrical energy and data from the canal unit 10 is under the scope of the present subject matter, for example transmission of optical energy and the like. FIG. 6 illustrates a canal unit 10 comprising a canal optical transmitter 109 instead of the canal transmitting antenna 108. Any type of optical transmitter is under the scope of the present subject matter, for example light emitting diode (LED) and the like. Accordingly, the receiving antenna 152 functions as an optical receiver that is configured to receive the optical energy and transform the optical energy to electrical energy. Any type of optical receiver is under the scope of the present subject matter, for example a photoelectric cell and the like.

According to one embodiment, the canal unit 10 is configured to be divided to multiple parts, wherein the multiple parts are configured to reassemble to form a complete canal unit 10.

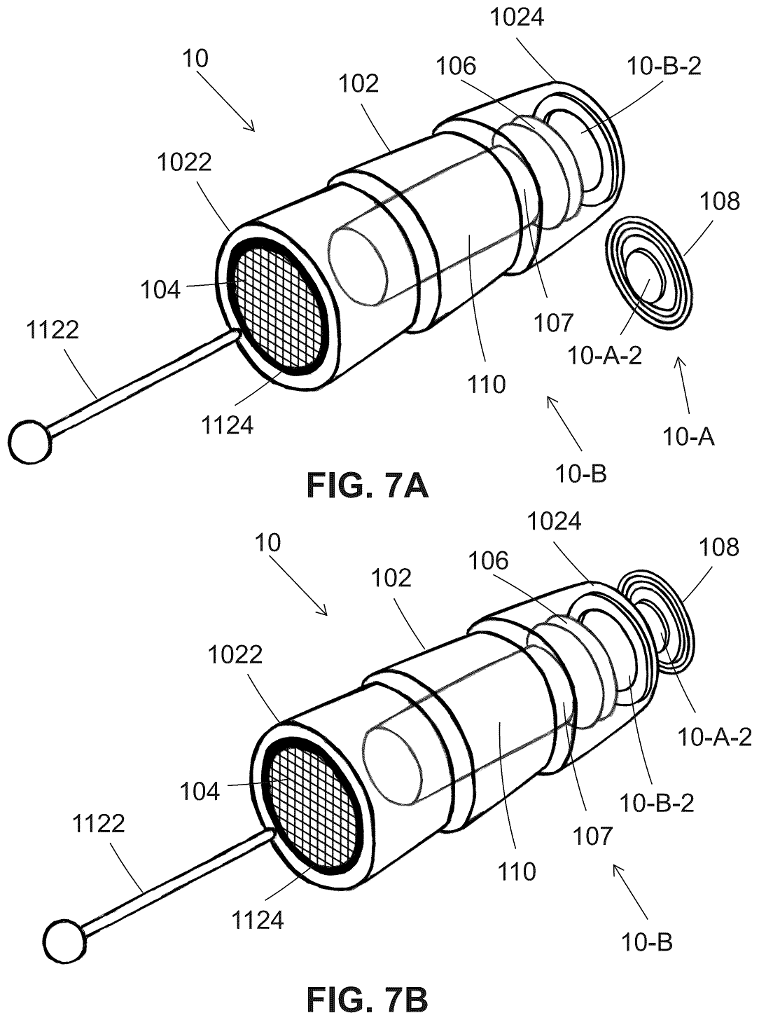

Referring now to FIG. 7A schematically illustrating, according to an exemplary embodiment of the present subject matter, a perspective see-through view of a canal unit of a hidden cochlear implant system divided into two parts.

Referring now to FIG. 7B schematically illustrating, according to an exemplary embodiment of the present subject matter, a perspective see-through view of a canal unit of a hidden cochlear implant system divided into two parts that are partially reassembled.

According to one embodiment, the components of the canal unit 10 can be distributed in any combination between the parts of the canal unit 10, wherein the parts are divided one from the other. The embodiment illustrated in FIGS. 7A-B is exemplary only and should not be considered as limiting the scope of the present subject matter. As can be seen in FIG. 7A, the canal unit 10 is divided into two parts-a first canal unit part 10-A, and a second canal unit part 10-B. According to another embodiment, the first canal unit part 10-A comprises the canal transmitting antenna 108, while the second canal unit part 10-B comprises the other components of the canal unit 10 described above, namely at least the at least one microphone 104, the canal modulator 106, and the canal electrical power source 110. As can be seen in FIGS. 7A-B, the second canal unit part 10-B can further comprise additional components of the canal unit 10. Thus, during insertion of the canal unit 10 into the ear canal 940 of a user, the first canal unit part 10-A can be inserted firstly and positioned in the ear canal 940, adjacent to the ear drum 950. Then, the second canal unit part 10-B can be inserted into the ear canal 940 and reassembled with the first canal unit part 10-A, as shown in FIG. 7B. In order to facilitate the reassembly of the parts of the canal unit 10, the canal unit 10 can further comprise, according to some embodiments, mechanisms for attaching the parts one to the other. For example, as can be seen in FIG. 7A, a mechanism for attaching the parts one to the other can be a male-female mechanism. Thus, the first canal unit part 10-A can comprise a male member 10-A-2, and the second canal unit part 10-B can comprise a female member 10-B-2 that is configured to attach to the male member 10-A-2. Of course, an opposite orientation of the male and female members is also under the scope of the present subject matter.

Referring now to FIG. 8 schematically illustrating, according to an exemplary embodiment of the present subject matter, a perspective view of internal and external components of a human ear and a canal unit inserted in the ear canal.

FIG. 8 illustrating a canal unit 10 inserted in the ear canal 940 of an ear 9 of a user. The canal unit 10 that is illustrated in FIG. 8 comprises inter alia a handle 1122. However, it should be noted that this embodiment of the canal unit 10 inserted in the ear canal 940 is only exemplary, and should not be considered as limiting the scope of the present subject matter. Any embodiment of the canal unit 10, described herein, is similarly configured to be inserted in the ear canal 940.

As can be seen in FIG. 8, the outward side 1022 of the canal unit 10 points toward the pinna 910. As a result, the at least one canal microphone 104 residing at the outward side 1022 of the canal unit 10 can easily receive sound signals that enter into the ear canal 940. In addition, an at least one component configured to facilitate grasping of the canal unit 10, for example a handle 1122, an attractable element 1124, or a combination thereof, that reside at the outward side 1022 of the canal unit 10, can be easily grasped, or attracted by an appropriate tool that is inserted into the ear canal 940 through the pinna 910. As can further be seen in FIG. 8, the inward side 1024 of the canal unit 10 points toward the ear drum 950 and the cochlea 920. As a result, the canal transmitting antenna 108 residing at the inward side 1024 of the canal unit 10 can transmit electrical energy, for example in a form of a wireless carrier signal, and data, for example in a form of a modulated wireless carrier signal, toward the ear drum 950 and the cochlea 920.

Referring now to FIG. 9 schematically illustrating, according to an exemplary embodiment of the present subject matter, a side view of an implanted unit of a hidden cochlear implant system, the implanted unit comprising a processor that is configured to be implanted in the vicinity of the cochlea.

Referring now to FIG. 10 schematically illustrating, according to an exemplary embodiment of the present subject matter, a perspective view of internal components of a human ear and an implanted unit implanted in the vicinity of the cochlea, the implanted unit comprising a processor that is configured to be implanted in the middle ear in the vicinity of the cochlea.

According to one embodiment, at least part of the implanted unit 15 is configured to be implanted in a cochlea 920 of an ear 9 of a user. According to another embodiment, the implanted unit 15 comprises a cochlear receiving antenna 152, a processor 154 and an electrode array 156, wherein the cochlear receiving antenna 152 is electrically connected to the processor 154, and the processor 154 is electrically connected to the electrode array 156.

According to one embodiment, the cochlear receiving antenna 152 is configured to receive electrical energy, for example in a form a wireless carrier signal, and data, for example in a form of a modulated wireless carrier signal, that is wirelessly transmitted by the canal transmitting antenna 108, convert the wireless carrier signal to an electrical carrier signal, and transmit the electrical carrier signal to the processor 154. The wireless carrier signal can be modulated; or not modulated, namely be only electrical energy, as described above. According to one embodiment, the cochlear receiving antenna 152 can have any shape known in the art. According to a preferred embodiment, the cochlear receiving antenna 152 has a coil-like shape. According to another preferred embodiment, the cochlear receiving antenna 152 is configured to be implanted in the middle ear in the vicinity of the ear drum 950 aside the cochlea 920.

According to one embodiment, the processor 154 is configured to receive a modulated electrical carrier signal from the cochlear receiving antenna 152, demodulate the modulated electrical carrier signal to produce electrical sound signals, and transmit the electrical sound signals to the electrode array 156. According to one embodiment, the processor 154 is in a form of a chip, for example an A SIC chip, as known in the art. Thus, the processor 154 is further configured to perform additional tasks relating to controlling the function of the hidden cochlear implant system 1, and ensuring proper function of the hidden cochlear implant system 1. Some exemplary functions of the processor 154 include error check of decoded electrical sound signals to ensure proper decoding, controlling the timing and direction of transmission of the decoded electrical sound signals, and the like.

According to one embodiment, the processor 154 is configured to be implanted in any place, as desired.

According to one embodiment, illustrated in FIG. 10, the processor 154 is configured to be implanted in the vicinity of the ear drum 950 aside the cochlea 920. According to another embodiment, the processor 154 is configured to be implanted in the middle ear in the vicinity of the cochlea 920, aside the cochlear receiving antenna 152.

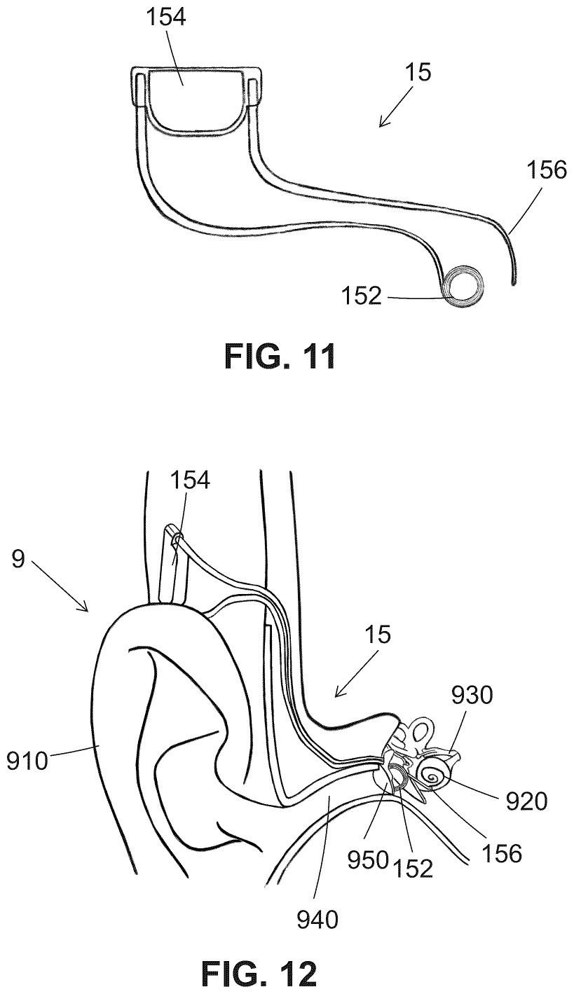

Referring now to FIG. 11 schematically illustrating, according to an exemplary embodiment of the present subject matter, a perspective view of an implanted unit comprising a processor that is configured to be implanted under the skin, in the vicinity of the pinna, on the mastoid bone.

Referring now to FIG. 12 schematically illustrating, according to an exemplary embodiment of the present subject matter, a perspective view of internal components of a human ear and an implanted unit at least partially implanted in the vicinity of the cochlea, the implanted unit comprising a processor that is configured to be implanted under the skin, in the vicinity of the pinna, on the mastoid bone.

According to one embodiment, illustrated in FIGS. 11 and 12, the processor 154 is configured to be implanted under the skin, in the vicinity of the pinna 910, on the mastoid bone. This embodiment is preferred for at least two reasons. Firstly, the surgical procedure of implanting a processor under the skin, in the vicinity of the pinna 910, on mastoid bone, and usage of such a processor are currently known in the art. This ensures that the hidden cochlear implant system 1 of the present subject matter would function properly, since according to this embodiment it employs a processor and surgical procedure that are already known and reliable. Another advantage is that the components of the hidden cochlear implant system 1 are hidden, and therefore not recognizable.

According to one embodiment, the electrode array 156 is configured to receive electrical sound signals from the processor 154, and stimulate the auditory nerve 930 with these electrical sound signals. According to another embodiment, the electrode array 156 is configured to be in contact with the auditory nerve 930. Any type of electrode array 156 known in the art, that is configured to stimulate the auditory nerve 930 with electrical sound signals, is under the scope of the present subject matter, for example an electrode configured to transmit electrical pulses, a vibrating electrode configured to stimulate the auditory nerve 930 by vibrations, and the like.

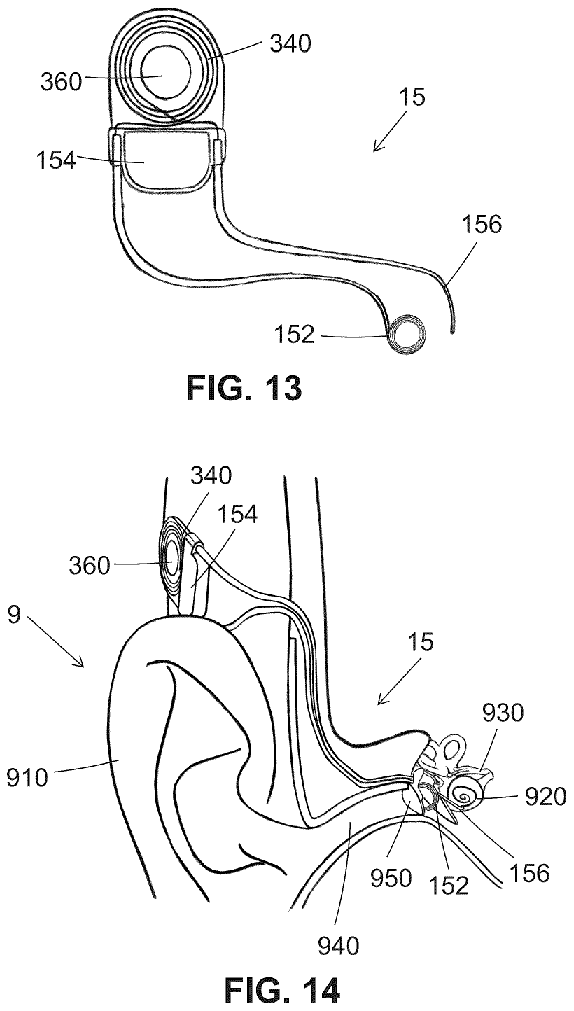

Referring now to FIG. 13 schematically illustrating, according to an exemplary embodiment of the present subject matter, a side view of another embodiment of an implanted unit of a hidden cochlear implant system.

Referring now to FIG. 14 schematically illustrating, according to an exemplary embodiment of the present subject matter, a perspective view of internal components of a human ear and another embodiment of an implanted unit that is implanted partially in the vicinity of the cochlea, and partially implanted under the skin, in the vicinity of the pinna, on the mastoid bone.

As mentioned above, according to one embodiment, the implanted unit 15 is configured to receive electrical energy, for example in a form of a wireless carrier signal, and data, for example in a form of a modulated wireless carrier signal, from the canal unit 10, and eventually stimulate the auditory nerve 930 with electrical sound signals as described above, given that the wireless carrier signal is modulated. According to another embodiment, the implanted unit 15 is further configured to receive electrical energy, for example in a form of a wireless carrier signal, and data, for example in a form of a modulated wireless carrier signal from an external source such as transmitting antenna 330 of a prior art cochlear device 3, and eventually stimulate the auditory nerve 930 with electrical sound signals as described above.

According to one embodiment, the implanted unit 15 that is configured to receive electrical energy and data from the canal unit 10 and from source such as the external transmitting antenna 330 can be implanted in a user that has not been using any type of cochlear implant before.

One advantage of the implanted unit 15 that is configured to receive electrical energy, for example in a form of a wireless carrier signal from the canal unit 10 and from the external transmitting antenna 330 is that it allows a user to switch between usage of a canal unit 10 and between an external microphone 310, an external modulator 320, and an external transmitting antenna 330. Thus, for example when a user cannot use the canal unit 10, for example because of an ailment in the ear canal 940, the user can still be able to hear sound and speech by using the external microphone 310, the external modulator 320, and the external transmitting antenna 330. Another advantage of the implanted unit 15 that is configured to receive electrical power and data from the canal unit 10 and from the external transmitting antenna 330 is that the implanted unit 15 that is configured to receive electrical power and data from the canal unit 10 and from the external transmitting antenna 330 allows switching to a hidden cochlear implant system 1 of the present subject matter, without a need to remove components of the prior art cochlear implant 3.

It should be noted that the functions of the components of the implanted unit 15 that is configured to receive electrical energy from the canal unit 10 and from the external transmitting antenna 330 and the interrelations of the components of the implanted unit 15 that is configured to receive also a wireless carrier signal from the canal unit 10 and from the external transmitting antenna 330, as well as their positions in the ear 9 and in the vicinity of the ear 9, are similar to those described above. Therefore, only a brief description of the implanted unit 15 that is configured to receive electrical energy and data from the canal unit 10 and from the external transmitting antenna 330 would be given hereinafter, in combination with FIGS. 13 and 14.

According to one embodiment, the implanted unit 15 that is configured to receive electrical energy and data from the canal unit 10 and from the external transmitting antenna 330 comprises a cochlear receiving antenna 152 that is preferably placed in the middle ear, an internal receiving antenna 340 that is preferably placed in proximity of the mastoid bone, a processor 154, and an electrode array 156. The cochlear receiving antenna 152 is electrically connected to the processor 154, the internal receiving antenna 340 is electrically connected to the processor 154, and the processor 154 is electrically connected to the electrode array 156. According to another embodiment, the implanted unit 15 that is configured to receive electrical energy and data from the canal unit 10 and from the external transmitting antenna 330 can further comprise an internal magnet 360.

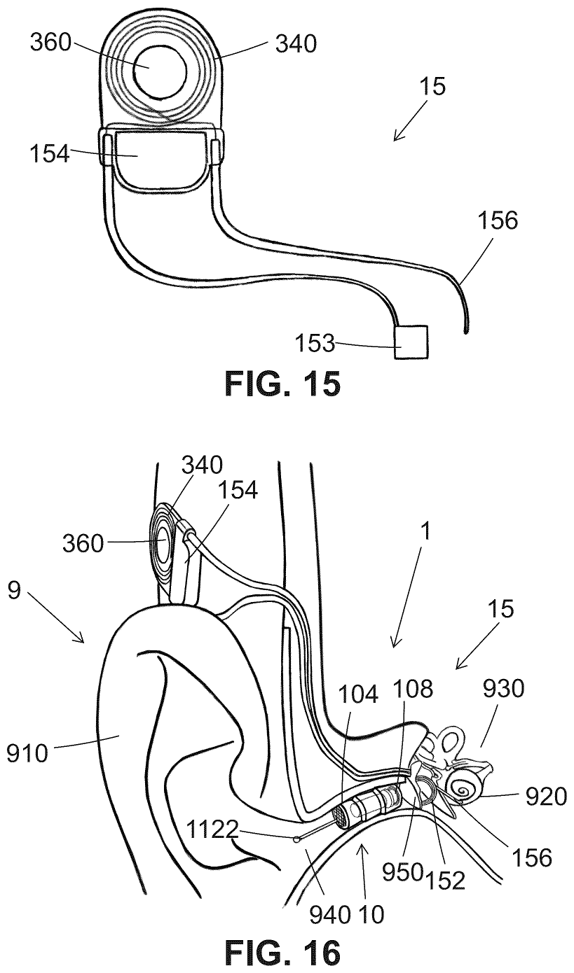

As described above, the canal transmitting antenna 108 is configured to transmit electrical energy and data, and the cochlear receiving antenna 152 is configured to receive the electrical energy and data. Any type of wireless communication, as well as transmission of electrical energy and data, between the canal transmitting antenna 108 and the cochlear receiving antenna 152 is under the scope of the present subject matter. For example, usage of electromagnetic radio signals, and usage of short-wavelength ultra-high frequency radio waves—a technology known as “Bluetooth”. Another example is usage of optical energy transmission. According to this example, the canal transmitting antenna 108 is of a type of an optical transmitter 109, as illustrated in FIG. 6, for example an optical transmitter in a form of a LED, and the cochlear receiving antenna 152 is of a type of an optical receiver 153, as illustrated in FIG. 15, for example in a form of a photoelectric cell.

Referring now to FIG. 15 schematically illustrating, according to an exemplary embodiment of the present subject matter, a perspective see-through view of a side view of an implanted unit of a hidden cochlear implant system, further comprising a cochlear optical receiver instead of the cochlear receiving antenna.

Even not shown, it should be noted that the cochlear optical receiver 153 can replace also the cochlear receiving antenna 152 of the implanted unit 15 that is illustrated, for example, in FIG. 9.

Referring now to FIG. 16 schematically illustrating, according to an exemplary embodiment of the present subject matter, a perspective view of internal components of a human ear and an implanted unit implanted partially in the vicinity of the cochlea, and partially implanted under the skin, in the vicinity of the pinna, on the mastoid bone, as well as a canal unit inserted in the ear canal.

FIG. 16 illustrates a human ear 9, where an implanted unit is implanted according to embodiments described above, and a canal unit 10 is inserted in the ear canal 940 according to embodiments described above. This embodiment is advantageous, as described above, since it allows a user to use either the canal unit 10, or the external components of the prior art cochlear implant 3.

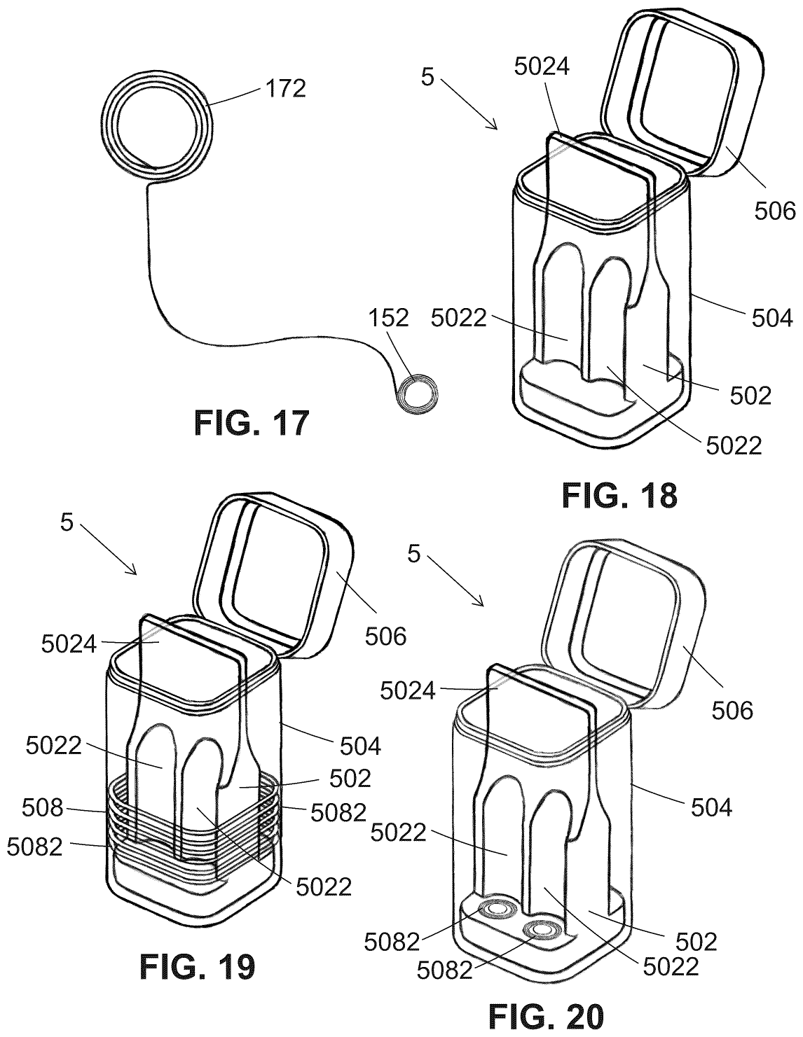

Referring now to FIG. 17 schematically illustrating, according to an exemplary embodiment of the present subject matter, a side view of an internal receiving antenna adaptor electrically connected to a cochlear receiving antenna.

According to one embodiment, the internal receiving antenna adaptor 172 is configured to be connected to the internal receiving antenna 340. Thus, according to a preferred embodiment, the shape of the internal receiving antenna adaptor 172 is similar to the shape of the internal receiving antenna 340, for example a coil-like structure, as shown in FIG. 17, when the internal receiving antenna 340 has a coil like structure. According to another embodiment, the internal receiving antenna adaptor 172 is electrically connected to the cochlear receiving antenna 152. According to yet another embodiment, the internal receiving antenna adaptor 172 is configured to receive electrical energy and data from the cochlear receiving antenna 152 and transmit electrical energy and data to the internal receiving antenna 340, that is connected to the internal receiving antenna adaptor 172. This embodiment is useful for a user that has already a prior art cochlear implant 3, and desires to use a canal unit 10 instead of the external components of the prior art cochlear implant 3. Thus, the internal receiving antenna adaptor 172 that is electrically connected to a cochlear receiving antenna 152, as illustrated in FIG. 17, is implanted in the user's ear, in a manner that the cochlear receiving antenna 152 is implanted in the middle ear in the vicinity of the cochlea 920 aside the ear drum 950, as shown for example in FIG. 10, while the internal receiving antenna adaptor 172 is connected to the internal receiving antenna 340. Thus, after inserting the canal unit 10 into the ear canal 940, electrical energy and data are received by the cochlear receiving antenna 152, transmitted to the internal receiving antenna adaptor 172, and from there to the internal receiving antenna 340. Then the electrical energy and data 4 continue their path as described in relation to the prior art cochlear implant 3.

According to some embodiments, the canal unit 10 is configured to be inserted into the ear canal 940 until the inward side 1024 of the canal unit 10 is in close vicinity to the ear drum 950, more particularly, until the canal transmitting antenna 108, that resides at the inward side 1024 of the canal unit 10, is in close vicinity to the ear drum 950. According to another embodiment, the canal unit 10 is configured to be inserted into the ear canal 940 until the canal transmitting antenna 108 is in an optimal distance from the cochlear receiving antenna 152 that resides in the middle ear on the other side of the ear drum 950. This embodiment is of importance during the process of insertion of the canal unit 10 into the ear canal 940 of a user, because in one hand it is necessary to avoid damage to the ear drum 950 when, for example, the canal unit 10 is inserted too deep into the ear canal 940; and on the other hand it is necessary that the distance between the canal transmitting antenna 108 and the cochlear receiving antenna 152 is optimal, in order get an optimal transmission of signals between the two antennae.

Thus, according to one embodiment, the hidden cochlear implant system 1 is configured to determine the distance between the canal transmitting antenna 108 and the cochlear receiving antenna 152. According to another embodiment, the hidden cochlear implant system 1 is configured to produce an alarm signal when the distance between the canal transmitting antenna 108 and the cochlear receiving antenna 152 reaches a predetermined value. Alternatively, according to yet another embodiment, the hidden cochlear implant system 1 is configured to produce an alarm signal as long as the distance between the canal transmitting antenna 108 and the cochlear receiving antenna 152 is not similar to a predetermined value.

Any method known in the art for determining a distance between a transmitting antenna and a receiving antenna is under the scope of the present subject matter.

One example is transmission of short pulses by the canal transmitting antenna 108, and measurement of the rate of decay of the pulses received by the cochlear receiving antenna 152. As the distance between the canal transmitting antenna 108 and the cochlear receiving antenna 152 is shorter, the rate of decay of the pulses is higher. Thus, a correlation between the decay rate and the distance between the two antennae can be determined or calculated, and used for determining the distance between the antennae, for example during insertion of the canal unit 10 into the ear canal 940.

Another example is letting the canal transmitting antenna 108 to transmit signals in various different frequencies, and determine the frequency that produces the highest voltage in the cochlear receiving antenna 152. This frequency producing the highest voltage is the common resonance frequency of the two antennae. The common resonance frequency depends on the distance between the two antennae. Thus, a correlation between the resonance frequency and the distance between the two antennae can be determined, and used for determining the distance between the antennae, for example during insertion of the canal unit 10 into the ear canal 940.

It should be noted, though, that the aforementioned examples for determining the distance between the canal transmitting antenna 108 and the cochlear receiving antenna 152 should not be considered as limiting the scope of the present subject matter, and that any method for determining the distance between the two antennae is under the scope of the present subject matter.

In addition, any mechanism known in the art for producing an alarm signal according to the aforementioned embodiments, is under the scope of the present subject matter. For example, the hidden cochlear device 1 is configured to produce a sound alarm; a vibration alarm; a light alarm; a wireless alarm that includes transmission of a signal to a computing member, like a computer, a smartphone and the like, when the signal provokes the computing device to produce an alarm; and the like. Accordingly, the hidden cochlear device 1 further comprises at least one alarm signal producer known in the art, as should be understood by a person skilled in the art.

Referring now to FIGS. 18-20 schematically illustrating exemplary embodiments according to the present subject matter of a storing member.

The present subject matter further provides a storing member 5. According to one embodiment, the storing member 5 is configured to store at least one canal unit 10. According to another embodiment, the storing member 5 is configured to protect at least one canal unit 10 that is stored in the storing member 5, against physical impacts, dirt and the like. According to yet another embodiment, the storing member 5 is configured to charge the canal electrical power source 110 of the at least one canal unit 10 that is stored in the storing member 5, given that the canal electrical power source 110 is rechargeable.

According to one embodiment, illustrated for example in FIG. 18, the storing member 5 comprises a base 502 configured to accommodate at least one canal unit 10, and a case 504 configured to accommodate the base 502. According to another embodiment, the storing member 5 further comprises a cover 506 configured to cover the case 504, prevent accidental exit or removal of the at least one canal unit 10, or of the base 502 from the case 504, and in addition prevent entrance of dirt into the case 504 and cause damage to the at least one canal unit 10.

According to one embodiment, the base 502 comprises at least one niche 5022, each niche 5022 configured to accommodate a canal unit 10. According to another embodiment, each niche 5022 is configured to tightly grasp the canal unit 10, in order to avoid accidental release, or falling, of the canal unit 10 from the niche 5022.

According to one embodiment, the base 502 further comprises a handle 5024 configured to be grasped by a user, in order to facilitate insertion of the base 502 into the case 504, removal of the base 502 from the case 504, and carrying the base 502.

According to one embodiment, illustrated in FIGS. 19-20, the storing member 5 further comprises an electricity charging element 508 configured to charge the canal electrical power source 110 of the at least one canal unit 10 that is stored in the storing member 5, given that the canal electrical power source 110 is rechargeable. Any type of electricity charging element 508 known in the art is under the scope pf the present subject matter. Two exemplary embodiments of the electrical charging element 508 are illustrated in FIGS. 19-20.

According to one embodiment, illustrated in FIG. 19, the electricity charging element 508 comprises multiple electrical wires 5082 surrounding the base 502, and the at least one canal unit 10 accommodate on the base 502. The multiple electrical wires 5082 are connected to an electrical power source, for example a mains electricity (not shown), as can be easily understood by a person skilled in the art. Electric power is transferred by electrical induction from the multiple electrical wires 5082, through a metal coil in the canal unit 10, to the canal electrical power source 110. An exemplary metal coil can be the canal transmitting antenna 108 of the canal unit 10, having a coil-like structure.

According to another embodiment, illustrated in FIG. 20, the electricity charging element 508 comprises multiple electrical wires 5084 positioned on a bottom of each niche 5022 of the base 502. A canal unit 10 is placed in a niche 5022 when the canal transmitting antenna 108 faces the bottom of the niche 5022 and the multiple electrical wires 5084. The multiple electrical wires 5084 are connected to an electrical power source, for example a mains electricity (not shown), as can be easily understood by a person skilled in the art. Electric power is transferred by electrical induction from the multiple electrical wires 5084, through the canal transmitting antenna 108, given that the canal transmitting antenna 108 has a coil-like structure, to the canal electrical power source 110.

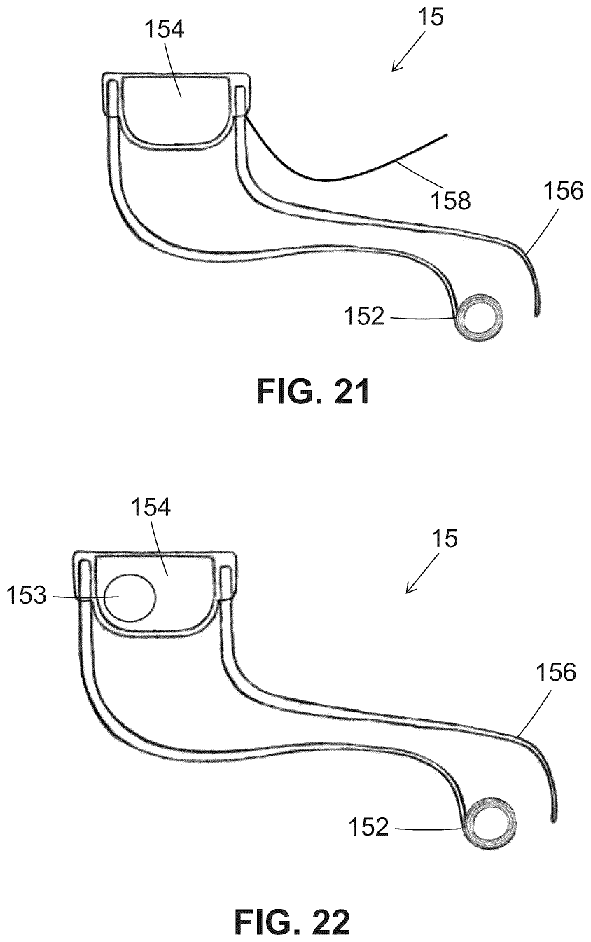

Referring now to FIG. 21, schematically illustrating, according to an exemplary of the present subject matter, a side view of another embodiment of an implanted unit of a hidden cochlear implant system further comprising a ground.

According to one embodiment, the implanted unit 15 further comprises a ground 158, configured to prevent damage to the brain in case of a short circuit, as known in the art. The other components illustrated in FIG. 21 are similar to the components shown in FIG. 11.

Referring now to FIG. 22 schematically illustrating, according to an exemplary of the present subject matter, a side view of another embodiment of an implanted unit of a hidden cochlear implant system further comprising an implant electrical power source.

According to one embodiment, the implanted unit 15 further comprises an implant electrical power source 153. Any type of electrical power source known in the art can serve as an implant electrical power source 153. For example, the implant electrical power source 153 can be a battery, a rechargeable battery, and the like. According to another embodiment, the implant electrical power source 153 is electrically connected to any component of the implanted unit 15 that requires supply of electrical power, for example the processor 154, and the like. The other components illustrated in FIG. 21 are similar to the components shown in FIG. 11.

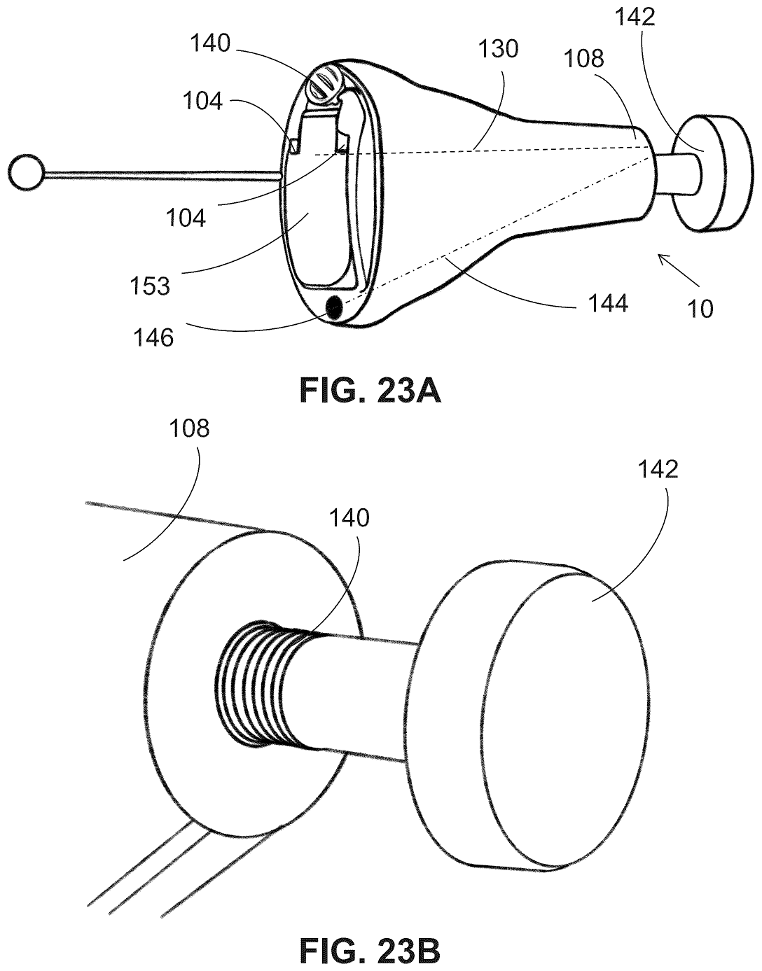

Referring now to FIG. 23A schematically illustrating, according to an embodiment of the present subject matter, a view of a canal unit of the hidden cochlear implant system. The hidden cochlear implant system comprises a canal unit 10 that allows adjustment of a distance between the canal transmitting antenna 108 and a receiving antenna (not shown in this figure). For example, in order to personalize the hidden cochlear implant system to dimensions of the ear of individual users. For example, the canal processor 107 (not shown in FIG. 23A) is configured to determine the distance between the canal transmitting antenna 108 and the receiving antenna (such as receiving antenna 152 shown as example in FIGS. 10 and 16) using a software that calculates the distance between the canal transmitting antenna 108 and the receiving antenna according to a difference between the level of energy that is transmitted from the canal transmitting antenna 108 and the level of energy received by the receiving antenna. According to the amount of energy gap, the software calculates how much energy is absorbed by the receiving antenna 152 and translates this value into the distance between the canal transmitting antenna 108 and the receiving antenna. According to the output of the software, the distance between the canal transmitting antenna 108 and the receiving antenna can be adjusted in order to optimize the transfer of energy from the canal transmitting antenna 108 to the receiving antenna.

According to one embodiment, the hidden cochlear implant system is configured to adjust the distance between the canal transmitting antenna 108 and the receiving antenna (152, as an example). According to an exemplary embodiment, the canal unit 10 comprises a distance adjuster 130 (shown in dash line since it is preferably an internal mechanism) that is configured to change a position of the transmitting antenna 108, preferably by pulling and pushing in the canal unit 10, thereby adjusting the distance between the canal transmitting antenna 108 and the receiving antenna. An exemplary distance adjuster 130 is a screw 130, as shown in FIG. 23A that is connected to the transmitting antenna 108 at one side of the screw. Screwing the screw 130, for example with a corresponding screwdriver, changes the position of the transmitting antenna 108 in the canal unit 10. For this purpose, a head 140 of the screw 130 is positioned on a side of the canal unit 10 that is directed towards the outer side of the ear canal 940 in order to allow access to the screw 130 with a screwdriver. Another exemplary distance adjuster 130 is a motor for example an electric motor that is configured to change the position of the transmitting antenna 108 in the canal unit 10 as described above.

According to an additional embodiment, canal unit 10 comprises a ventilation channel 144 (shown by a dash line). The ventilation channel 144 communicates the interior part of the ear with the exterior part. Canal unit 10 can reside inside user's ear canal for a long time, and in order to allow air to flow into the canal space, a hollow channel can be used. Optionally, hollow channel that is part of the ventilation channel 144 is in the same perimeter as the canal unit 10. The ventilation channel 144 starts at the interior side 146 of canal unit 10 and reaches the external end thus allowing passage of air. The ventilation canal 144 can be located in several locations along the canal unit 10, for example, having the ventilation channel 144 at the lower end of the canal unit fits the style of a completely in canal (CIC) processor. The ventilation channel can also comprise filters, wax blocker, valves and other parts.

Referring now to FIG. 23B schematically illustrating, according to an embodiment of the present subject matter, a partial view of a canal unit of a hidden cochlear implant system. According to one embodiment, the hidden cochlear implant system is configured to adjust the distance between the canal transmitting antenna 108 and the receiving antenna 152. Adjusting mechanism 140 is configured to change the distance between the canal transmitting antenna 108 and the receiving antenna 152. According to this embodiment, the canal transmitting antenna 108 is attached to a moving part 142 that is capable of moving towards the receiving antenna 152. Moving part 142 comprises a screw having a thread like end that comprises a tapering groove that spirals towards or away from the receiving antenna (not shown in this figure).

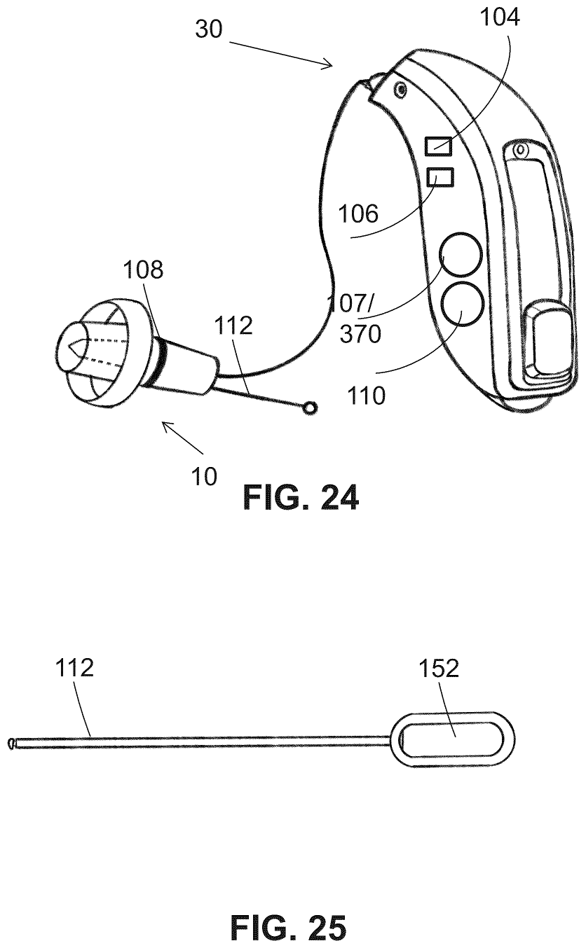

Referring now to FIG. 24 schematically illustrating, according to an embodiment of the present subject matter, an implant system in which some of the implant system components are configured to reside in an external unit. According to the embodiments described above, the canal unit 10 comprises all the components of the canal unit described above, meaning that all these components reside in the ear canal 940. According to some other embodiments, some of these components can reside in an external unit 30 except of the transmitting antenna 108 and in some embodiments the grasping element 112 that must reside in the ear canal 940. For example, each of the microphones 104, modulator 106, processor 370, electric power source 110 can reside in an external unit 30. Each of the other components can reside either in the canal unit 10 or in the external unit 30. The components that reside in the external unit are electrically connected to the components that reside in the canal unit 10, for example, wirily or wirelessly.

The external unit 30 is configured to be positioned externally to the ear. Preferably, the external unit 30 is placed behind the ear. For example, to the pinna or to any item convenient to a user. An advantage of this embodiment is related, for example, to the electrical power source. Placing the electrical power source in the external unit 30 allows usage of a larger electrical power source having a longer battery life and a larger electrical capacity than in a case in which the electrical power source is implanted.

Referring now to FIG. 25 schematically illustrating, according to an embodiment of the present subject matter, a view of a receiving antenna. According to one embodiment, the receiving antenna 152 is elliptically shaped thus facilitating insertion of the receiving antenna 152 into the middle ear, which resides between the ear drum 950 and the cochlea 920, and can more conveniently being inserted during surgery using the grasping element 112.

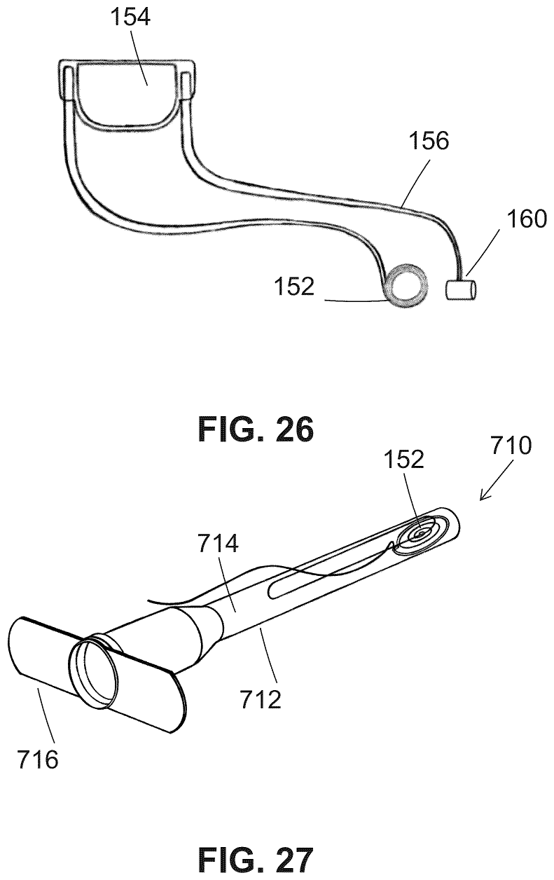

Referring now to FIG. 26 schematically illustrates, according to an exemplary embodiment of the present subject matter, a perspective view of an implanted unit comprising a processor that is configured to be implanted under the skin, in the vicinity of the pinna, on the mastoid bone. According to one embodiment, the electrode array 156 is configured to receive electrical sound signals from the processor 154, and stimulate the auditory nerve with these electrical sound signals. According to another embodiment, the electrode array 156 is configured to be in contact with the auditory nerve. Vibrating electrode array 160 is configured to stimulate the auditory nerve by vibrations.

Referring now to FIG. 27 schematically illustrating, according to an embodiment of the present subject matter, a view of an exemplary conduit. According to another embodiment, the hidden cochlear implant system further comprises a conduit 710 configured to facilitate implementation of at least one component as described above of the implanted unit. For example, in order to facilitate implementation of the receiving antenna 152, or the entire implanted unit in the middle ear.

The conduit 710 comprises an elongated hollow element 712 comprising a lumen 714, and a pushing element 716 configured to be inserted into the lumen 714 of the elongated hollow element 712 and push at least one component of the implanted unit (such as implanted unit 15, as shown in FIG. 9 as an example) placed in the lumen 714. For implanting the implanted element in the middle ear, at least one component of the implanted unit, for example the receiving antenna 152, is placed inside the lumen 714 of the elongated hollow element 712. Then, the elongated hollow element 712 is brought to the vicinity of the middle ear and the at least one component of the implanted unit is pushed into the middle ear using the pushing element 716.

According to one embedment the receiving antenna 152 is configured to be inserted into the lumen 714 of the elongated hollow element 712, and pushed out of the elongated hollow element 712 into the middle ear during implementation. According to another embodiment, the size and shape of the receiving antenna 152 facilitates insertion of the receiving antenna into the lumen 714. According to yet another embodiment, the receiving antenna 152 is made of a material having a shape memory, for example silicone, namely during insertion of the receiving antenna 152 into the lumen 714, the size and shape of the receiving antenna 152 is adapted to the dimensions of the lumen 714, and after implanting, the receiving antenna 152 in the middle ear, the receiving antenna 152 assumes its original size and shape.

In order to better understand the preferred embodiments, the electrical energy and data transmission of the described hidden cochlear implant system will be explained as follows: The electrical energy and data transmission is referred to in this document as in canal wireless transmission system (ICWTS). The ICWTS is referring to wireless transmission of electrical energy and data from an external antenna in the ear canal to a receiving antenna implanted in the ear. The electrical energy that is wirelessly transmitted is used as power to drive the electrical components of the implant, and practically, acts as a power source for the implanted unit. Preferably, the transmission is an electromagnetic transmission using two coils as will be shown hereinafter. The data transmission between the units can be modulated on the power carrier transmission; but can also be transmitted in any way known in the art such as bluetooth low energy (BLE) and the like.

According to a preferred embodiment, the ICWTS has similar properties such as the hidden cochlear implant system describe herein before and as an example, cochlear receiving antenna 152 that was described hereinbefore, and can be located in several positions. One of the preferred locations is in the mastoid cavity, near the ear canal inner wall facing the ear canal direction. As mentioned before, the receiving antenna 152 receives power and data from the in canal transmitting antenna 108.

More specifically, it is provided a ICWTS that is associated with an implant that is configured to stimulate an organ in a head of a user having an ear and an ear canal, the ICWTS comprises a canal transmitting antenna to be positioned within the ear canal, wherein the canal transmitting antenna is configured to transmit electrical energy; and an implanted receiving antenna to be implanted in the ear, wherein the implanted receiving antenna is connected to the implant and receives the electrical energy that functions as a power source.

An extension for this embodiment is to have an additional receiving antenna 340 located in the mastoid cavity facing the outer side of the head that receives power and data from an external transmission antenna 320 that is located on the head. The power and data transmission between the additional receiving antenna 340 and the external transmission antenna 320 is called an external transmission system. The combination option of those systems will be called hidden transmission system.

According to the preferred embodiment, the hidden transmission system includes a unidirectional wireless transmission of electrical energy and bidirectional wireless transmission of data communication, which means that in the ICWTS the power is transmitted from the external in canal antenna to the receiving antenna in the ear, while data can be transmitted and received from both antennas. For example, sound data that is collected by the canal microphone is transferred to the implanted unit by the in canal transmitting antenna through the cochlear receiving antenna. Then, the implanted unit can send feedback to the external unit by the cochlear receiving antenna through the in canal transmitting antenna. Similarly, in the external transmission system, the power is transmitted from the external antenna to the receiving antenna in the ear, while data can be transmitted and received from both antennas. sound data that is collected by the external microphone is transferred to the implanted unit by the external transmitting antenna through the additional receiving antenna. Then, the implanted unit can send feedback to the external unit by the additional receiving antenna through the external transmitting antenna.

Data from the receiving antenna can be transferred to the transmitting antenna by forming small changes in the resonance circuit by switching loads or capacitor that will form changes in the inductive link. The changes are detected by the transmitter circuit since changes in the coil current are due to inductive changes. The changes are translated as data.

It can be understood from some of the embodiments herein described that one of the challenges in designing a hidden cochlear implant system is obtaining the best position between the transmitting antenna in the canal unit and the receiving antenna in the implanted unit that is effective and efficient in wirelessly transmitting power and data. There are several parameters that need to be considered when the physician is positioning the antennas. Traditional hearings aids are positioned between the aperture of the ear canal and the area between the isthmus and the actual second bend of the ear canal. Further, insertion of the device deeper in the canal may cause discomfort to the wearer and the process of taking a deep impression must be done with care to prevent injury. Insertion of the device deeper in the canal can be done using some methods that require a professional doctor to insert a deeper device or an alarm that informs the wearer that the device is correctly positioned.

There are several traditional hearings aids as follows: behind the ear (“BTE”) hearing aids for the treatment of mild to moderately severe hearing loss that rely on acoustic domes (soft silicon ear tips) attached to a receiver in the canal (RIC) which are mounted no more than 0.5 cm past the aperture of the ear canal opening. In the ear (ITE) devices are positioned between the concha of the ear and the first bend of the ear canal. In the canal (ITC) hearing aids are positioned somewhat similarly between the lower portion of the cochlea and the first bend of the ear canal. Completely in the canal (CIC hearing aids are positioned between the aperture of the ear canal and the area between the isthmus and the actual second bend of the ear canal. The rationale for the very deep position for CIC hearing aids is based primarily on the prevention of acoustic feedback and providing the necessary acoustic gain.

First, placement of the receiving antenna will be discussed. According to one aspect of the present subject matter, the receiver coil is placed in the mastoid cavity, close to the ear canal inner wall. This approach has several advantages as follows:
The space that can accommodate the receiver antenna is larger, a fact that increases the possibilities to orient the coil in a most effective way.
The distance between the transmitting and the receiving coils is shorter than any other distance between the coils when the receiving coil is located in different places such as the middle ear or under the skin on the mastoid bone. The ear canal wall has a small thickness and is the main separating organ between the coils.
The surgery procedure during which the coils are placed is a traditional cochlear implant (CI) surgery in which the mastoid cavity is exposed and allows easy attachment of the receiving coil to the inner wall of the ear canal.
Achieving better and safer placement of the transmission coil that is located in the ear canal laterally to the receiving coil. The placement is similar to the placement of custom in-the-ear (ITE) or in-the-canal (ITC) hearing aids without deep canal insertion. In this way, a bigger battery can be used in the ITE and ITC form factor.
There is no need for special alarms for the patients and the audiologist since there is no risk of pushing the external device too deep inside the ear canal and no risks associated with very deep ear impression taking.

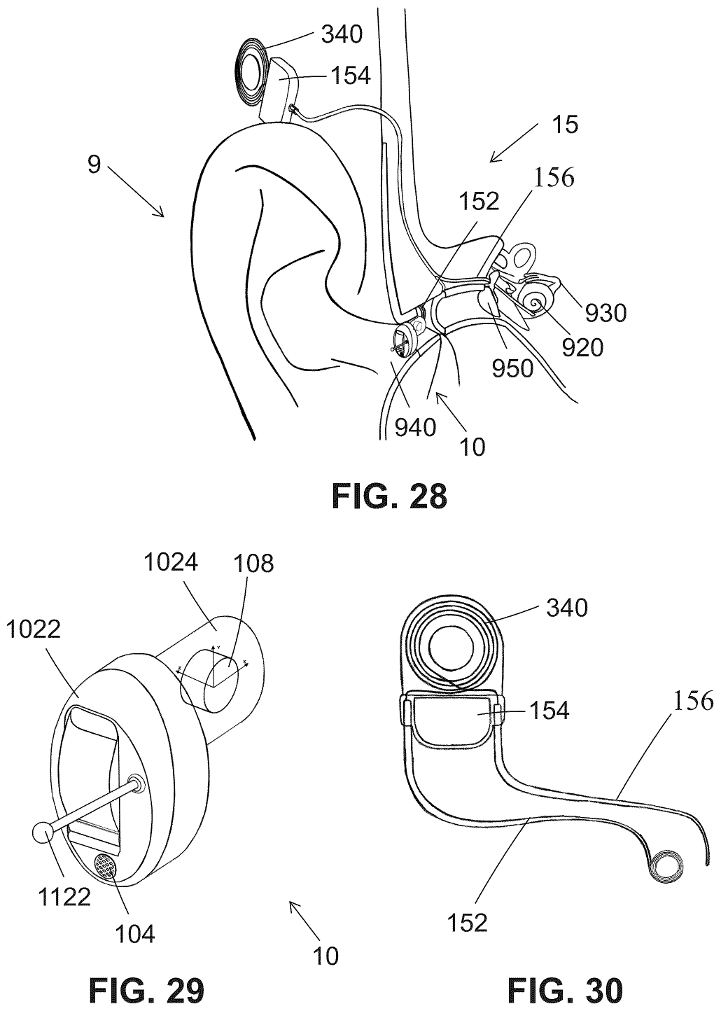

Reference is now made to FIG. 28 schematically illustrates, according to an exemplary embodiment of the present subject matter, a perspective view of internal components of a human ear and an implanted unit when the electrode array is implanted in the cochlea, and the coils and processor are implanted in the ear, in the mastoid cavity, as well as a canal unit inserted in the ear canal.

Reference is made to FIG. 29 schematically illustrates, according to an exemplary embodiment of the present subject matter, a perspective see-through view of a canal unit of a hidden cochlear implant system.

Reference is made to FIG. 30 schematically illustrates, according to an exemplary embodiment of the present subject matter, a perspective view of an implanted unit to be partially implanted within the mastoid cavity.

FIG. 28 illustrates ICWTS in a cochlear implant with a canal unit 10 inserted in the ear canal 940 of an ear 9 of a user as an in canal transmitter and an implanted unit 15 that includes the receiver antenna 152 in the mastoid cavity, near the ear canal inner wall facing the ear canal direction and an additional receiver coil in the mastoid for the external transmission system. The canal transmitter is housed within a housing. There is a resemblance of the canal unit components to the embodiments described hereinbefore; however, the canal unit 10 in this embodiment is not inserted into the ear canal 940 as deep as in the previous shown embodiments. The transmitter coil is directed to the receiver coil that is positioned in the mastoid cavity or bone. The canal unit 10 itself can better be observed in FIG. 29 and comprises at the outward side 1022 of the unit, a microphone 104 that is configured to receive sound signals that enter the ear canal 940 and optionally, a handle 1122 that assists the user to position the canal unit in an optimal positioning.

On the other side of the canal unit 10, the inward side 1024 points toward the ear drum 950 and the cochlea 920 (shown in FIG. 28), when inserted to within the canal. The canal transmitting antenna 108 is positioned within the inward side 1024 while facing the mastoid cavity towards the direction of the cochlear receiving antenna. The direction of the transmitting antenna 108 in space is indicated by an earth centered inertial. As a result, the canal transmitting antenna residing at the inward side 1024 of the canal unit 10 can be designed to be thinner in its elongated axis than in the previous shown canal units. In such configuration, the shape of the transmitting antenna has better efficiency in the sense that the electromagnetic wave facing the mastoid cavity is better transferred to the receiving antenna wherein electrical energy as power is transmitted toward the receiver coil in the mastoid cavity.

As mentioned in the explanation to other embodiment hereinbefore, the implanted unit 15 is configured to receive electrical energy, for example in a form of a wireless carrier signal that carries power to drive the electrical components of the implant, and data, for example in a form of a modulated wireless carrier signal, from the canal unit 10, and eventually stimulate the auditory nerve 930 with electrical sound signals as described above, given that the wireless carrier signal is modulated. The data is transmitted on top of the power transmission and the implanted unit 15 is eventually configured to stimulate the auditory nerve 930 with electrical sound signals.

According to one embodiment shown in FIG. 28 and in more details in FIG. 30, the implanted unit 15 that is configured to receive electrical energy and data from the canal unit 10 using the ICWTS as described and optionally from an external source using the external transmission system as described. In FIG. 28, it can be seen that the receiving antenna 152 is implanted in the mastoid cavity near the ear canal wall, in very close proximity to the inward side 1024 of the canal unit 10 and the transmitting antenna 108 within. This close proximity has the advantages as discussed hereinbefore. An internal receiving antenna 340 is preferably placed in proximity of the mastoid bone facing the outer side of the head, adjacent to a processor 154. An electrode array 156 is placed in the cochlea. The cochlear receiving antenna 152 as well as the internal receiving antenna 340 are electrically connected to the processor 154, and the processor 154 is electrically connected to the electrode array 156.

As described above, the canal transmitting antenna 108 is configured to transmit power and data, and the cochlear receiving antenna 152 is configured to receive the electrical energy (power) and data. Any type of wireless communication, as well as transmission of electrical energy and data, between the canal transmitting antenna 108 and the cochlear receiving antenna 152 is under the scope of the present subject matter. For example, usage of electromagnetic radio signals, and usage of short-wavelength ultra-high frequency (UHF) radio waves-a technology known as “Bluetooth”. The electromagnetic coils transmission can also be referencing as inductive coil system, such as in canal inductive coils system. Another example is usage of optical energy transmission.

As described above, the canal transmitting antenna in the ICWTS is configured to transmit power and data, and the cochlear receiving antenna is configured to receive the electrical energy (power) and the data. Optionally, the receiver coil can be located in the tragus or in the helix area.

The advantage of this approach is as follows: The outer ear is made up of cartilage and skin and there is enough space to place the receiver coil onto the cartilage. Also, this is a very protected placement to locate the receiver coil. This approach creates an even smaller distance between the receiver coil and the transmission coil. In this way, better placement of the transmission coil is achieved. The transmission coil should be located laterally in the ear canal similar to the placement of a custom ITE or ITC hearing aids without a deep canal insertion.

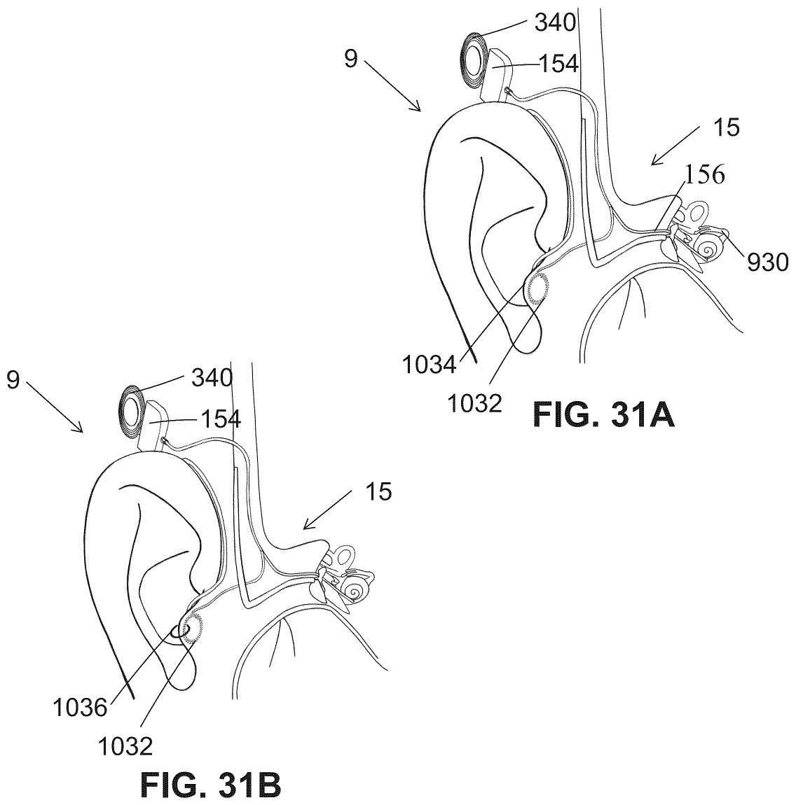

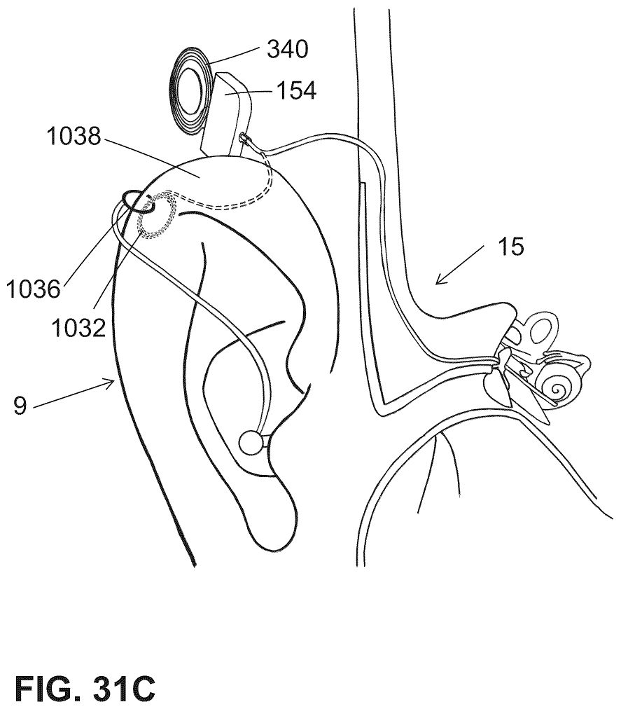

Reference is now made to FIG. 31A-31C schematically illustrate, according to an exemplary embodiments of the present subject matter, perspective views of an outer ear with an implanted unit and a receiving coil attached to it and positioned in the vicinity of the tragus, a perspective view of the shown outer ear with a receiving coil pierced, and a perspective view of an outer ear with an implanted unit and a receiving coil positioned in the helix with a pierced transmitting coil, respectively.

According to another embodiment of the present subject matter, the receiving antenna of the ICWTS is configured to be implanted within a tragus of the ear and the transmitting antenna is configured to be placed within the ear canal directed towards the receiving antenna.

As can be seen in FIG. 31A, the implanted unit 15 is similar in its principles to the previous embodiments, however, the receiving antenna 1032 is located under the skin in the vicinity of the tragus 1034 in the ear 9. The electrode array 156 that is configured to receive electrical sound signals from processor 154, and stimulates the auditory nerve 930. A transmitting antenna is provided in an in-canal unit that is not shown in this figure. Other elements such as microphone or power source can implement with any type of transmission external devices. In FIG. 31B, a transmitting coil 1036 is shown to be pierced onto the tragus 1034 of an ear 9. The implementation of implanting the receiving coil 1032 in the tragus is surgically more complicated than a regular placement of the receiving coil in the mastoid. Implanting the receiving coil in the tragus or the helix, as examples, requires expanding the surgery area in order to locate the coil in place. The transmission coil 1036 has to be closer to the battery, a positioning that can cause electromagnetic effects and loss of energy through the transmission. In order to overcome this disadvantage, a relatively big ferrite is used that controls the electromagnetic field and prevent mutual effect from the battery. In that position, the transmission coil 1036 is located outside the ear canal and close to the tragus in the outer ear.

As mentioned herein before, the receiving coil can be placed in any placement on the outer ear 9, where there is cartilage and skin such as the helix, the antihelix, and even on the Lobule. In accordance with another embodiment shown in FIG. 31C, the receiving coil 1032 is implanted in the vicinity of the helix 1038 of the ear 9 while the transmitting coil 1036 is pierced within the helix 1038 in the vicinity of the receiving coil.

In the approach in which the receiving coil and the transmitting coil are attached to the tragus or to the helix, a transmitting coil can be pierced to the ear in a way by which it is interconnected to the receiving coil that is designed like a ring, as can be clearly seen in FIGS. 31B and 31C. This arrangement of the coils can dramatically affect the energy transmission efficiency and can provide a transmission efficiency of about 90%. Another advantage of this approach is that these locations in the ear are less likely to get infected. Accordingly, data is transferred from the transmission unit to the receiving unit. Optionally, each coil has its own RF signal monitor output, through which the processing unit 154 can accept the incoming data from the transmission unit. The separated incoming data lines allow the processing unit 154 to identify the active coil in the system. A DC energy storage capacitor provides power to the receiving unit while the transmission RF signal is inhibited.

In accordance with another embodiment, a hidden transmission system is provided that comprised an ICWTS and an external transmission system comprising an additional receiving antenna provided to the implant and located in a mastoid cavity facing an outer side of the head and an external transmitting antenna located on the outer side, wherein the additional receiving antenna receives power and data from the external transmission antenna.

Another aspect of the present subject matter is to provide an ICWTS incorporated within a bone conduction system commonly referred to as a bone anchored hearing aid (BAHA). Instead of sending sound signals through the ear canal, the BAHA transmits sound vibrations through the skull bone directly to the cochlea.

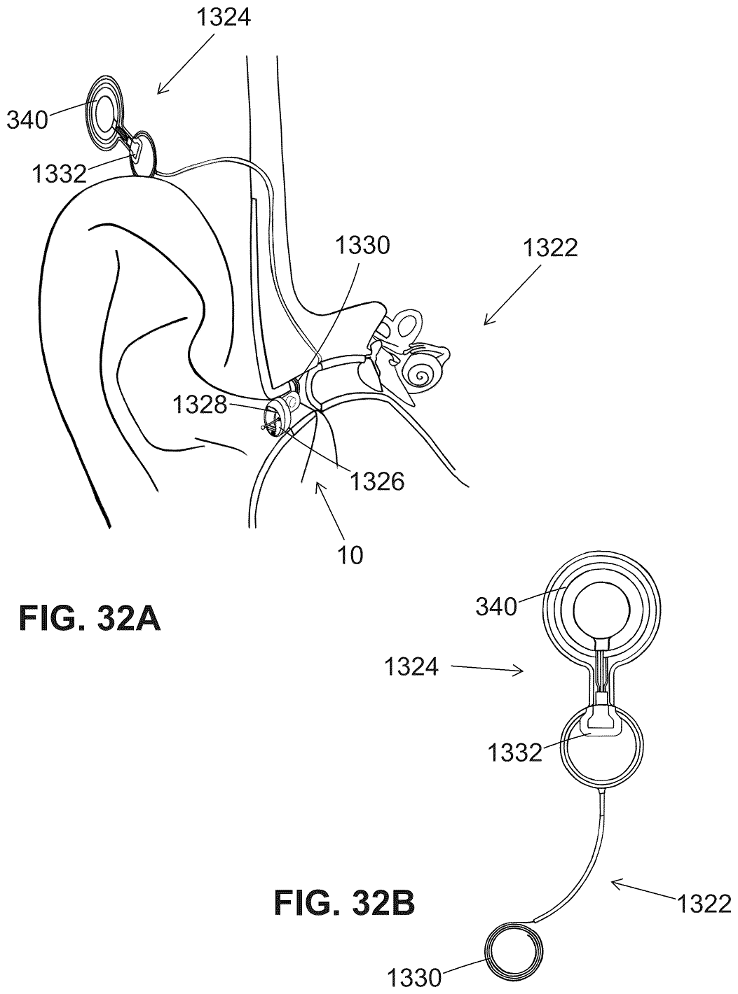

Reference is now made to FIG. 32A and FIG. 32B schematically illustrate, according to an exemplary embodiment of the present subject matter, a perspective view of an ICWTS incorporated within a BAHA partially implanted within an ear and an implanted unit incorporated within a BAHA, respectively.

The combined BAHA and ICWTS comprise two units: an implanted unit 1322 and an external unit 1324. A sound processor 1326 in a canal unit 10 converts the sound picked up by a microphone 1328, which is also located in the canal unit 10, into electrical data. Sound processor 1326 then transmits the electrical data to the implanted unit 1322 using electromagnetic energy transfer from a transmitting antenna in the canal, as explained hereinbefore. Then, the data received in the cochlear receiving antenna 1330, which is positioned in the mastoid bone, of the implanted unit 1322, converts the data into vibration. The BAHA is provided with a vibrator 1332 that vibrates and transfers signals through the bones of the skull, to the cochlea of the inner ear.

It is noted that the receiving antenna 1330 in this case is implanted within the mastoid bone near the canal wall as clearly seen in FIG. 32A. During the surgical procedure of implantation, a tunnel and a location for the coil is surgically formed. In case the canal unit 10 is removed, a receiving coil 340 is provided in the BAHA implant 1324 as an external transmission system describe before to receive the sound signals and transferred them into vibrations, as in a regular BAHA device.

The ICWTS can be implemented and be used for several applications. One of the examples is its utilization as a neurostimulation device such as deep brain stimulation (DBS) devices, or transcranial magnetic stimulation (TMS) devices. Neurostimulation devices are implantable, programmable medical devices that deliver electrical stimulation to specific parts of the patient's brain, spinal cord, or peripheral nervous system to help treat various conditions, including chronic pain, movement disorders, epilepsy and Parkinson's. Neurostimulation devices are built from an implanted unit that is configured to deliver electrical stimulation to specific locations. The implanted unit required power from an external source or from a battery. According to an embodiment of the present subject matter, the ICWTS can supply power or charging to the neurostimulation implanted device—the internal unit. The internal unit consists of a receiving coil that is located in the mastoid bone in accordance with the present subject matter, and an external unit that is located in the ear canal to provide power, as explained hereinbefore.

Another example is a neuro controlling system that monitors the brain and requires power supply to activate the device. The ICWTS can be used as a power supply to the neuro control system.

Optionally or additionally, the ICWTS can be incorporated with a vestibular implant, which is a medical device designed to restore or improve balance function in individuals with severe vestibular disorders. Such disorders can significantly impair a person's balance, leading to dizziness, vertigo, and difficulty in performing daily activities. The vestibular implant aims at providing artificial stimulation to the vestibular system, which is responsible for maintaining balance and spatial orientation. According to the present subject matter, an external processor is provided that is worn outside the body, usually behind the ear. The external processor captures motion data and converts it into electrical signals. An internal implant is surgically implanted inside the skull, near the vestibular organs, wherein the implant receives signals from the external processor and stimulates the vestibular nerve or organs. In a similar manner as previously described, an implanted receiving antenna and a transmitting antenna in a canal unit according to the present subject matter is integrated with the vestibular implant. Sound captured by a microphone in the canal unit is transmitted as electrical stimulation to the cochlea.

According to the method of the present subject matter, there is a balance between the use of a fully implantable device and the implantable device with an external unit. In accordance with another embodiment of the present subject matter, a fully implantable cochlear implant device that contains full cochlear implant components can be integrated with the ICWTS.

A fully implantable cochlear implant configured to be combined with an ICWTS as discussed hereinbefore so as to form a combined hidden transmission system, the combined hidden transmission system comprises a processor; a microphone functions as a main data source and collects sound; a power source; and an electrode array. The electrical energy transmitted from the canal transmitting antenna functions as an alternative power supply or as a charger to the power source and the data is transmitted from the canal transmitting antenna to the processor through the receiving antenna. For example, when the in canal unit is located in placed, both power and data are transmitted through the ear canal unit and not from the internal microphone and battery. When the ear canal unit is not in placed, the fully implantable cochlear implant receives power from the internal battery and sound from the internal microphone.

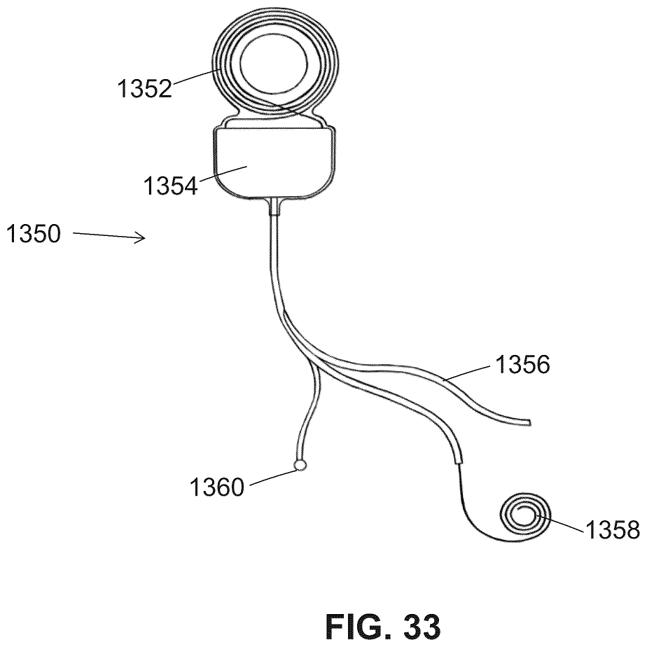

Reference is now made to FIG. 33 schematically illustrating a fully implantable unit, according to an exemplary embodiment of the present subject matter.

A fully implantable unit 1350 comprises the necessary parts of implant that are configured to be implanted within the ear portions and in the vicinity of the ear (the ear cannot be seen in FIG. 33). The fully implantable unit 1350 comprises an internal receiving antenna 1352 that is preferably implanted in proximity of the mastoid bone facing the outer side of the head, adjacent to a processor and battery unit 1354. An electrode array 1356 that is connected to the processor and battery unit 1354 and is implanted in the vicinity of the auditory nerve (not shown in this figure) so as to stimulate the nerve. The processor 1354 is further connected to a cochlear receiving antenna 1358 that is implanted in the mastoid cavity facing the in canal inner wall and to a microphone 1360 that is implanted in the vicinity of the skin so as to collect sound.

The fully implanted device is a self-sufficient device; however, it can be connected to the ICWTS that can be placed within the canal unit. In this case, the device can be switched so that the ICWTS will transmit signals to the implanted receiver coil. Upon removal of the ICWTS, in a regular mode, the processor and battery unit as well as the microphone are responsible to collect sound signals and transfer them to the implanted electrode array.

This approach benefits in its ability to hear or provide stimulation even when the external unit is not in place, in such cases when the external unit need to be recharge, expose to water, or during sleep. Several methods can be provided in order to detect whether the external unit is in place or removed:
i. Inductive link changes;
ii. Data transmission when the device is placed; and
iii. Sending an asking request from the internal unit in specific time, wherein if the request is answered, the device is connected. The request can be sent using any communication method known.

According to an aspect of the present subject matter, cochlear implants hybrids are a combination of cochlear implants nerve stimulation and hearing aids technology. This combination is meant for patients who have some level of acoustic hearing left. This system is a combination of the ICWTS according to the present subject matter and as described and an external device mounted on the ear. The external processor also consists of a speaker at the end of the unit, which functions as a hearing aid. This concept is suitable for patients with some level of hearing still functioning. In this way, the frequency that doesn't function is transferred using the stimulation from the implant, while the rest of the frequencies are sent using the speakers. Some of the patients may still have some degree of natural hearing. It is a substantial advantage to use the natural hearing that remains.

Methods for controlling the inductive circuits and changing parameters in real time can be implemented in the devices indicated in the present subject matter. Each inductive link has several parameters that are different between coils. Parameters such as frequency, resistors, capacitors, etc. For example, a frequency of 6.78M hz in one inductive coil system and 5M hz in the other. This change can facilitate differentiation between the coils without changing the circuits that drive them. The change of parameters should be performed in real time. The ability to change the coil capacitor in real time provides the option to easily find the frequency specific to the relevant coil. One of the advantages of this ability is to achieve the best efficiency of the system although there are small movements between the coils in daily life. Not like in the external transmission system, when the coils can be connected with a magnet, the in canal transmission system does not include a magnet, and the coils positioning is constantly changing. Implementing the capacitor change option in the devices of the present subject matter allow to control the efficiency of the devices. Another advantage is to be able to use two different coils in a single implant—the external transmission system and the in canal transmission system. In this way, change in the coils occurs without the need to change the circuits themselves and to support several frequencies with one circuit. For example, using a receiver antenna such as receiving coil 152 and receiving antenna such as receiving coil 340 in one implant while the in canal wireless transmission is working on 6.78 Mhz frequency and the external transmission system is working on 5 Mhz frequency. It should be noticed that the option to change the transmitter coil facilitates the use of the implantable device while keeping an external unit body with all the parameters in it such as microphone, DSP, and more.

In accordance with another embodiment of the present subject matter, an external unit (BTE) is comprises a plurality of socket connections configured to receive at least two transmission coils while the BTE can be switched from sending signals of one of the at least two transmission coils at a time.

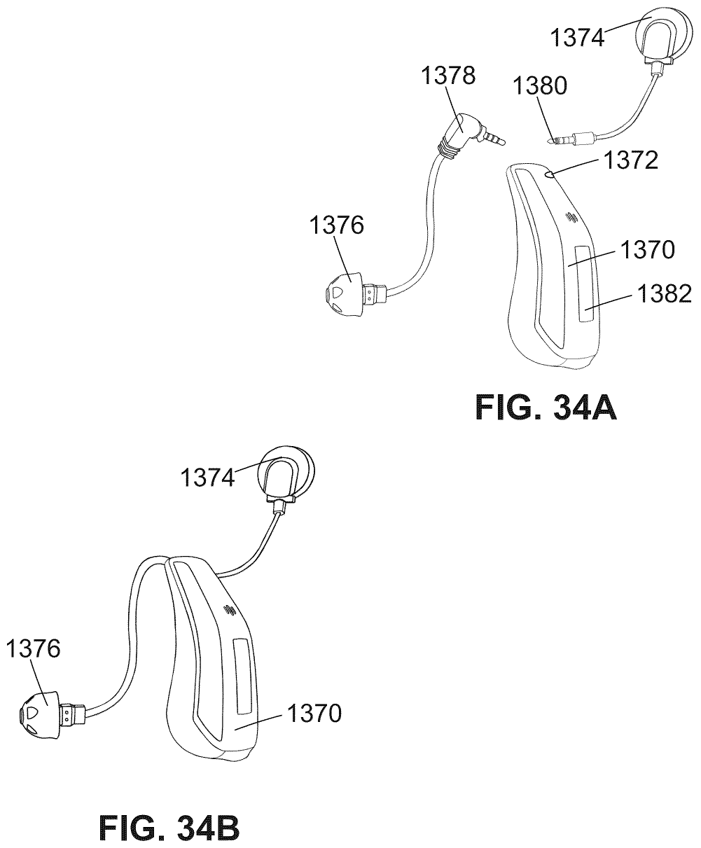

Reference is now made to FIGS. 34A and 34B schematically illustrating a BTE device configured to be connected to an ICWTS and an external transmission system, according to an exemplary embodiment of the present subject matter, and a connected system, respectively.

An external unit BTE 1370 has two sockets 1372 (only one of them can be observed in FIG. 34A) that are configured to receive connection of transmission coils of any type. As an example, a transmitting coil 1374 that is located on the head of the user, when in use, and another coil that can be provided in an in canal unit 1376, which optionally can be a ICWTS. Both the canal unit 1370 and the head transmitting coil 1374 can be plugs into the sockets 1372 using appropriate plugs 1378 and 1380. A switch 1382 is provided to the BTE 1370 that is configured to switch between the two connections. This can also be performed by putting the resonance circuit as an independent unit near the coil and can be disconnected and changed.

It is appreciated that certain features of the subject matter, which are, for clarity, described in the context of separate embodiments, may also be provided in combination in a single embodiment. Conversely, various features of the subject matter, which are, for brevity, described in the context of a single embodiment, may also be provided separately or in any suitable sub combination.

Although the subject matter has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

The invention claimed is:

1. An in-canal wireless transmitting system (ICWTS) associated with an implant that is configured to stimulate an organ in a head of a user having an ear and an ear canal, the ICWTS comprising:
a canal transmitting antenna to be positioned within the ear canal, wherein the canal transmitting antenna is configured to transmit electrical energy; and
an implanted receiving antenna to be implanted in the ear, wherein the implanted receiving antenna is connected to the implant and receives the electrical energy that functions as a power source.

2. The ICWTS as claimed in claim 1, wherein the ICWTS is further comprising a microphone and wherein the electrical energy further comprises sound signals received by the implanted receiving antenna and the organ is an auditory nerve.

3. The ICWTS as claimed in claim 1, wherein the implanted receiving antenna is positioned in a mastoid cavity in the ear, in a close proximity to an inner wall of the ear canal that is facing a direction of the ear canal.

4. The ICWTS as claimed in claim 3, wherein the canal transmitting antenna is directed to the implanted receiving antenna in the mastoid cavity.

5. The ICWTS as claimed in claim 1, wherein the canal transmitting antenna is transmitting unidirectional wireless transmission of electrical energy and bidirectional wireless transmission of data communication.

6. The ICWTS as claimed in claim 5, wherein the data communication is modulated on a power carrier transmission that transmits power.

7. A hidden transmission system comprising:
ICWTS as claimed in claim 1; and
an external transmission system comprising an additional receiving antenna provided to the implant and located in a mastoid cavity facing an outer side of the head and an external transmitting antenna located on the outer side,
wherein the additional receiving antenna receives power and data from the external transmission antenna.

8. The hidden transmission system as claimed in claim 7, further comprising a bone anchored hearing aid.

9. Fully implantable cochlear implant configured to be combined with an ICWTS as claimed in claim 3 so as to form a combined hidden transmission system, the combined hidden transmission system comprising:
a processor;
a microphone functions as a main data source and collects sound;
a power source; and
an electrode array,
wherein the electrical energy transmitted from the canal transmitting antenna functions as alternative power supply or as a charger to the power source and the data is transmitted from the canal transmitting antenna to the processor through the receiving antenna.

10. The combined hidden transmission system as claimed in claim 9, the system further comprises an external transmission system comprising an additional receiving antenna provided to the fully implantable cochlear implant and located in a mastoid cavity facing an outer side of the head and an external transmitting antenna located on the outer side of the head, wherein the additional receiving antenna receives power and data from the external transmission antenna.

11. A method of positioning the ICWTS as claimed in claim 1, the method comprising:
implanting the receiving antenna within a mastoid cavity in close proximity to an inner wall of the ear canal facing the ear canal, and
placing the transmitting antenna in the ear canal and directing the transmitting antenna towards the receiving antenna.

12. The ICWTS as claimed in claim 1, wherein the receiving antenna is configured to be implanted within a tragus of the ear and the transmitting antenna is configured to be placed within the ear canal directed towards the receiving antenna.

13. The ICWTS as claimed in claim 12, wherein the transmitting antenna is designed as a ring and is pierced so as to interconnect with the receiving antenna.

14. The ICWTS as claimed in claim 1 wherein the canal transmitting antenna and the receiving antenna are coils that function as inductive coils system transferring electrical energy and data using an electromagnetic transmission.

15. The ICWTS as claimed in claim 1, wherein the canal transmitting antenna is housed within a canal unit that is provided with an adjustment mechanism by which the position of the transmission antenna can be adjusted manually.

16. An external unit (BTE) comprises a plurality of socket connections configured to receive at least two transmission coils while the BTE can be switched from sending signals of one of the at least two transmission coils at a time.

17. The BTE of claim 16, wherein the one of the transmitting coils is a canal transmitting coil that is configured to be plugged in one of the plurality of sockets.
